(19) United States
(12) Patent Application Publication
(10) Pub. No.: US 2023/0167162 A1
(43) Pub. Date: Jun. 1, 2023

(54) HOMING PEPTIDE-GUIDED DECORIN CONJUGATES FOR USE IN TREATING EPIDERMOLYSIS BULLOSA

(71) Applicant: Tampere University Foundation sr, Tampere University (FI)

(72) Inventors: Tero Järvinen, Tampere University (FI); Toini Pemmari, Tampere University (FI); Ulrike May, Tampere University (FI)

(21) Appl. No.: 17/997,428

(22) PCT Filed: Apr. 28, 2021
(86) PCT No.: PCT/FI2021/050318

§ 371 Date: Oct. 28, 2022

Related U.S. Application Data
(60) Provisional application No. 63/016,359, filed on Apr. 28, 2020.

(30) Foreign Application Priority Data
Apr. 28, 2020 (FI) 20205431

(51) Int. Cl. C07K 14/47; A61P 17/02; A61K 47/64; C07K 5/11; C07K 7/06

(52) U.S. Cl. CPC C07K 14/4725; A61P 17/02; A61K 47/64; C07K 5/1019; C07K 7/06; C07K 2319/33; A61K 38/00

(57) ABSTRACT

The invention relates to a homing peptide-guided decorin conjugate for use in the treatment of epidermolysis bullosa, and to a corresponding method of treatment. Use of a novel homing peptide enables target-specific homing of the conjugate to skin and skin wounds in vivo, through systemic administration.

### TECHNICAL FIELD

The present invention relates generally to the field of molecular medicine. More specifically, the invention relates to a homing peptide-guided decorin conjugate for use in the treatment of epidermolysis bullosa, and to a corresponding method of treatment.

### BACKGROUND

Being the largest organ of the human body, skin presents unique challenges for efficient drug delivery. The primary challenge related to local, i.e. transdermal drug delivery is the poor penetration of macromolecules into the skin. Diffusion through intercellular lipids provides the option of transdermal delivery, but is limited only for small lipophilic molecules. Therefore, systemically administered, yet skin-specific therapeutics would be a substantial therapeutic advance for the treatment of skin diseases, particularly those that affect the entire skin, such as epidermolysis bullosa, a group of rare genetic diseases that cause fragile, blistering skin.

Recessive dystrophic epidermolysis bullosa (RDEB) is caused by mutations in COL7A1 gene that encodes type VII collagen (C7). Clinical manifestations include skin erosions and blistering, mutilating scarring, pseudosyndactyly and a high risk of developing aggressive and rapidly metastasizing cutaneous squamous cell carcinomas (cSCCs). Although some gene-, cell- and protein-based therapies have demonstrated promising results in delivering type VII collagen to the skin, challenges remain and there is still no cure for RDEB.

Transforming growth factor β (TGFβ) signaling has been demonstrated to play an essential role in the development of fibrosis and in the progression to malignancy in RDEB. Earlier, it has been demonstrated that TGFβ signaling is activated as early as a week after birth in col7a1^{-/-} mice (Liao et al, 2018, Stem Cells 36: 1839-1850). Thus, an early intervention on the activation of TGFβ signaling may be beneficial in reducing disease burden in RDEB. TGFβ signaling has also been suggested to be a phenotypic modulator in monozygotic twins with identical COL7A1 mutations (Odorisio et al., 2014, Hum Mol Genet 23: 3907-3922). Moreover, the expression level of a proteoglycan decorin (DCN), a natural TGFβ inhibitor, was significantly higher in the less affected twin. DCN is a structural constituent of extracellular matrix (ECM) and Dcn^{-/-} mice exhibit irregular collagen fibril formation and significantly reduced tensile strength in skin (Reed and Iozzo, 2002, Glycoconj J 19: 249-255). Furthermore, DCN has anti-fibrotic and anti-tumor functions by regulating activities of multiple growth factors, among them inhibitory action on TGFβ (Järvinen and Prince, 2015, Biomed Res Int 2015: 654765; Järvinen and Ruoslahti, 2019, Br J Pharmacol 176: 16-25). Recently, it has also been demonstrated an upregulation of DCN expression as one of the mechanisms of action for the effects of cord blood derived unrestricted somatic stem cells (USSCs) in col7a1^{-/-} mice (Liao et al., 2018, ibid.). Supporting the role of DCN as a potential therapeutic disease modifying molecule for RDEB, Cianfarani et al. (2019, Matrix Biol 81: 3-16) recently reported that systemic administration of lentivirus driving the expression of human DCN attenuated TGFβ induced fibrosis in C7-hypomorphic RDEB mouse model that expresses a residual level of type VII collagen (C7-hypomorphic mice).

Moreover, DCN binds and neutralizes connective tissue growth factor (CTGF/CCN2), which is a downstream mediator of TGFβ's fibrotic signaling and has been proposed to be a therapeutic target in prevention of scarring (Vial et al. 2011, J Biol Chem 286: 24242-24252; Daniels et al. 2003, Am J Pathol 163: 2043-2052). As the binding sites for TGFβ and CTGF/CCN2 reside in different parts of DCN, DCN theoretically can simultaneously block both mediators of fibrosis. Indeed, the role of DCN on suppressing TGFβ-driven scar formation has been well-established in numerous disease models such as renal, lung and hepatic fibrosis and in skin wound healing, in addition to RDEB (Odorisio et al., 2014, ibid.; Liao et al., 2018, ibid.; Cianfarani et al., 2019, ibid.). However, despite numerous positive anti-cancer and -fibrotic results in preclinical studies, DCN has not reached the clinic as systemic therapy. So far, the only reported clinical application of DCN was in 12 patients with perforating eye injury and a single dose of either 200 or 400 μg human recombinant DCN intravitreal injection appeared to be well tolerated with no ocular adverse events (Abdullatif et al., 2018, Graefes Arch Clin Exp Ophthalmol 256: 2473-2481).

A general limitation in systemic drug delivery is that only a small fraction of drug reaches its desired location and systemic side effects are encountered in other organs. Thus, a critical goal of modern drug development is to generate drugs to be target organ-specific, with minimal adverse effects in the other parts of the body. This goal could be achieved by developing drugs that recognize a specific epitope expressed in the affected organ. Alternatively, drugs can be converted to be target-specific by conjugation with an affinity ligand such as a vascular homing peptide that recognizes tissue- or target specific molecular features in the blood vessels in the given organ.

In vivo screening of phage peptide libraries has identified that these tissue or disease-specific molecular features in blood vessels (vascular zip codes) can be targeted by systemically administered affinity ligands such as vascular homing peptides. These studies have essentially established that organ or disease-specific molecular signatures in the vasculature of the different tissues exist, enabling a postal code system (vascular zip codes) for target-specific delivery of systemically administered therapeutics (Ruoslahti et al., 2010, J Cell Biol 188: 759-768; Ruoslahti, 2017, Adv Drug Deliv Rev 110-111: 3-12; Ruoslahti, 2004, Biochem Soc Trans 32: 397-402; Pasqualini and Ruoslahti, 1996, Nature 380(6572):364-366). The most efficient vascular homing peptides for tumor-specific homing and cell/tissue penetration contain a consensus motif R/KXXR/K (SEQ ID NO: 3), with an arginine (or rarely lysine) residue at the C-terminus, thus called C-end Rule (CendR) sequence (Ruoslahti, 2017, J Clin Invest 127: 1622-1624; Teesalu et al., 2009, Proc Natl Acad Sci USA 106: 16157-16162; Sugahara et al., 2009, Cancer Cell 16: 510-520; Sugahara et al., 2010, Science 328: 1031-1035). The CendR-sequence binds to neuropilin-1 (NRP-1), activating extravasation and tissue penetration pathway that delivers the peptide along with its payload into the parenchyma of the tumor tissue (Ruoslahti, 2017, Adv Drug Deliv Rev 110-111: 3-12; Ruoslahti, 2017, J Clin Invest 127: 1622-1624; Teesalu et al., 2009, PNAS 106(38):16157-16162). Peptides containing a cryptic CendR owe their target selectivity to combination of binding to a primary receptor with a tumor specific expression pattern, and to a proteolytic activation in the tumor to expose the CendR sequence in the target organ. As NRP-1 is expressed by the endothelial cells in all tissues (Ruoslahti, 2017, Adv Drug Deliv Rev 110-111: 3-12), the extravasation and tissue penetration via NRP-1 are unlikely to be restricted to cancerous tissues but happen in other diseased or healthy tissues as well.

In vivo phage display screens have also identified a panel of peptides that home to angiogenic blood vessels in skin wounds (Järvinen and Ruoslahti, 2007, Am J Pathol 171: 702-711) Two of the most promising peptides, cyclic peptides dubbed CAR (CARSKNKDC; SEQ ID NO: 5) and CRK (CRKDKC; SEQ ID NO: 3), have been utilized to deliver different therapeutic molecules in a target-selective fashion (Järvinen et al., 2017, ACS Biomaterials Science & Engineering 3: 1273-1282). Interestingly, although CRK peptide contains a cryptic CendR-sequence, RKDK (SEQ ID NO: 1), it is the only peptide among the vascular homing CendR peptides that is not capable of penetrating cells and tissues (Järvinen and Ruoslahti, 2007, Am J Pathol 171: 702-711; Agemy et al., 2010, Blood 116: 2847-2856).

WO 2008/136869 discloses the CRK peptide as a specific homing element for targeted delivery of decorin into skin wounds. The disclosed CRK-decorin fusions do not home to non-wounded skin.

Thus, systemically administered, yet skin-specific therapeutics would be a substantial therapeutic advance for the treatment of skin diseases, such as epidermolysis bullosa.

### SUMMARY

Provided herein is a homing peptide-guided decorin conjugate for use in the treatment of epidermolysis bullosa. The conjugate comprises a decorin segment and a homing peptide, wherein the C-terminal end of the homing peptide consists of the amino acid sequence RKDK (SEQ ID NO: 1) or CRKDK (SEQ ID NO: 2).

Also provided is a method of treating epidermolysis bullosa in a subject in need thereof by administering an efficient amount of a homing peptide-guided decorin conjugate comprising a decorin segment and a homing peptide, wherein the C-terminal end of the homing peptide consists of the amino acid sequence RKDK (SEQ ID NO: 1) or CRKDK (SEQ ID NO: 2).

Owing to the homing peptide, the conjugate selectively homes to and penetrates skin and skin wounds in vivo.

Embodiments and details of the above aspects are set forth in following figures, detailed description, examples, and dependent claims.

### BRIEF DESCRIPTION OF THE DRAWINGS

The accompanying drawings illustrate several embodiments of the disclosed subject matter, and together with the description, serve to explain principles of the disclosed compositions and methods.

FIGS. 1A to 1C illustrate the structure of an exemplary recombinant DCN-tCRK protein and its binding to neuropilin-1. FIG. 1A is a schematic representation of the structure of DCN-tCRK. Signal- and propeptide of the native DCN were replaced with a 6×His-tag (I) for purification. The His-tag is followed by the amino terminus (II), core protein (III), and carboxyl terminus (IV) of mature DCN proteoglycan. tCRK peptide (V) was cloned on the carboxyl end of the protein. FIG. 1B shows in vitro binding of DCN-tCRK to neuropilin-1 (NRP-1) in vitro. DCN-tCRK (left panel) and peptide controls (right panel, positive peptide: RPARPAR (SEQ ID NO: 25) and negative peptide: RPARPARA (SEQ ID NO: 26)) were immobilized in ELISA plate. Bovine serum albumin (BSA) was included as a non-specific protein control for DCN-tCRK and the peptides.

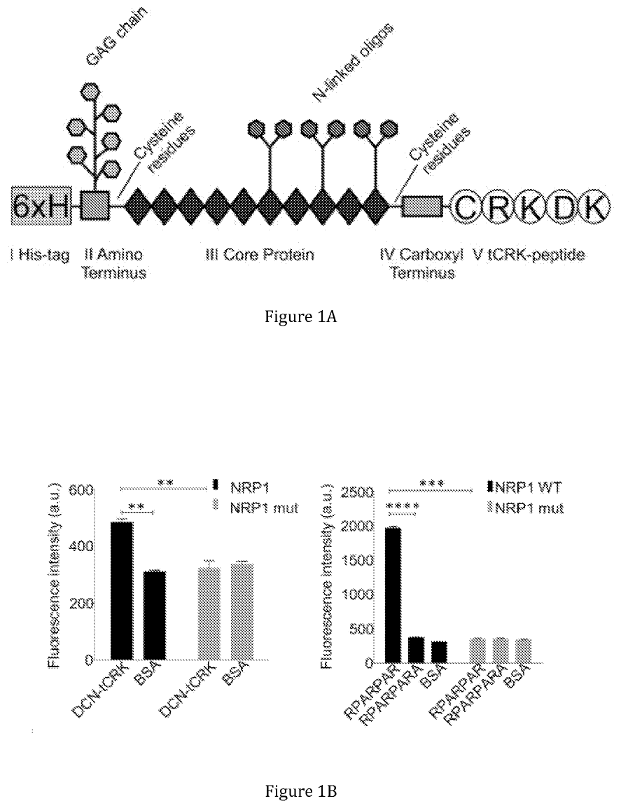

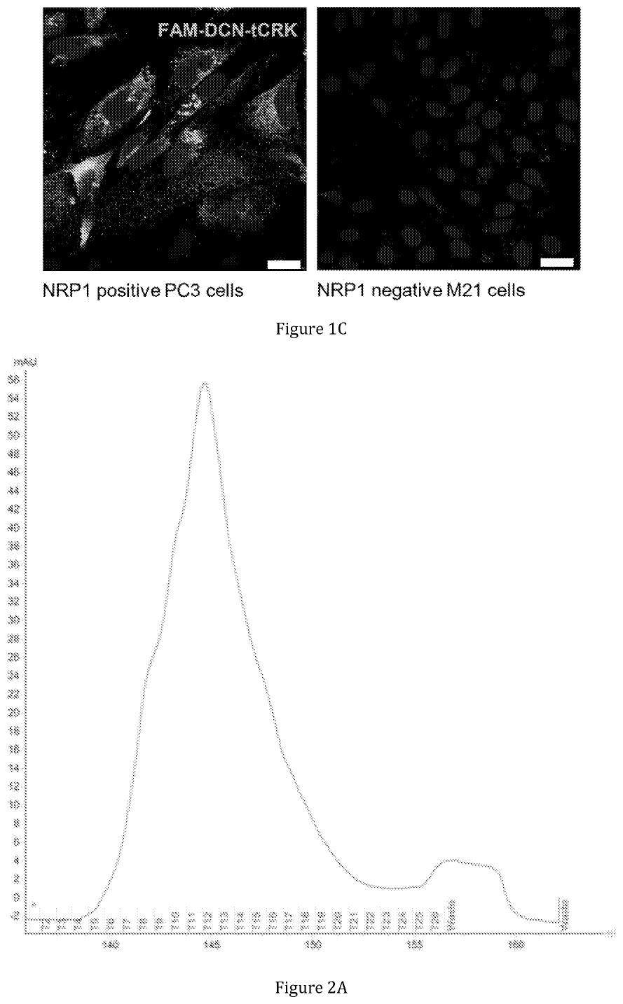

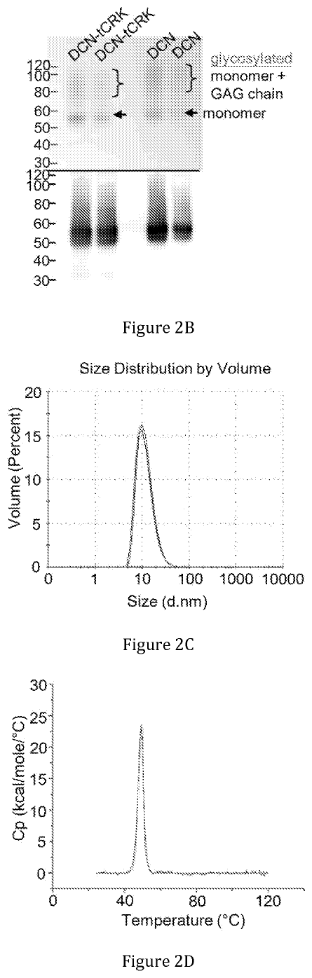

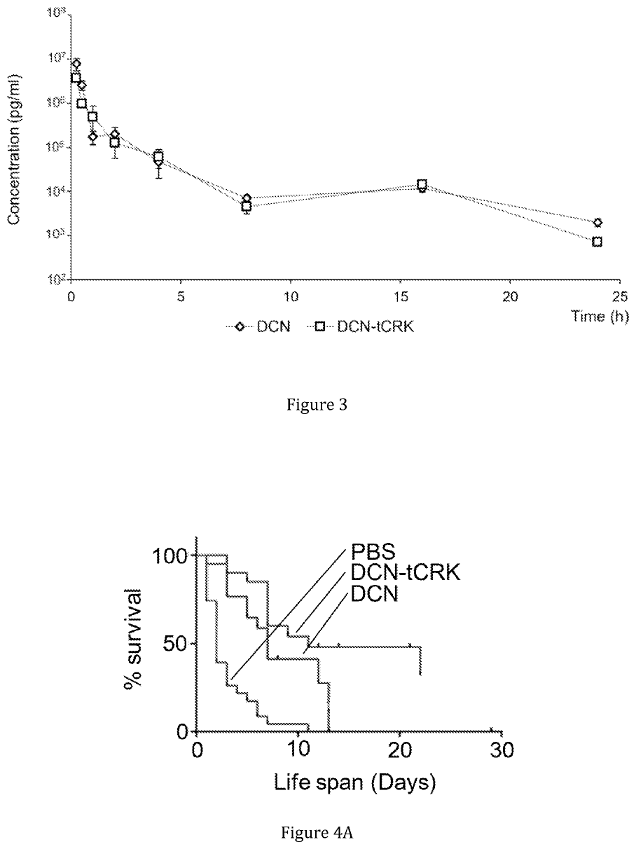

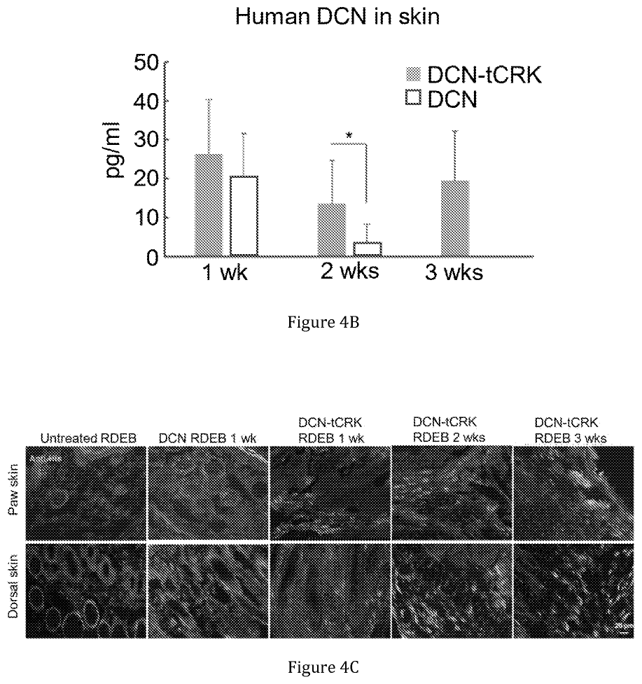

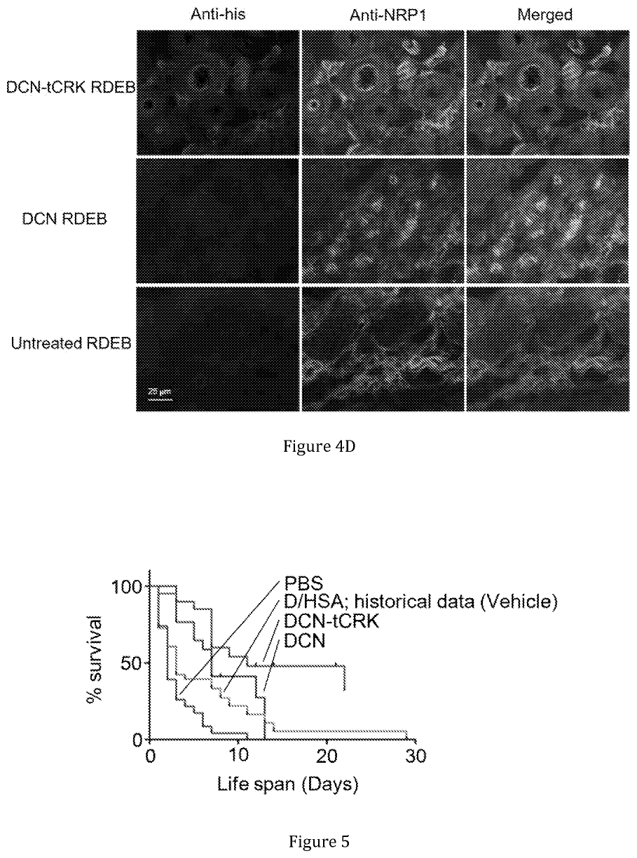

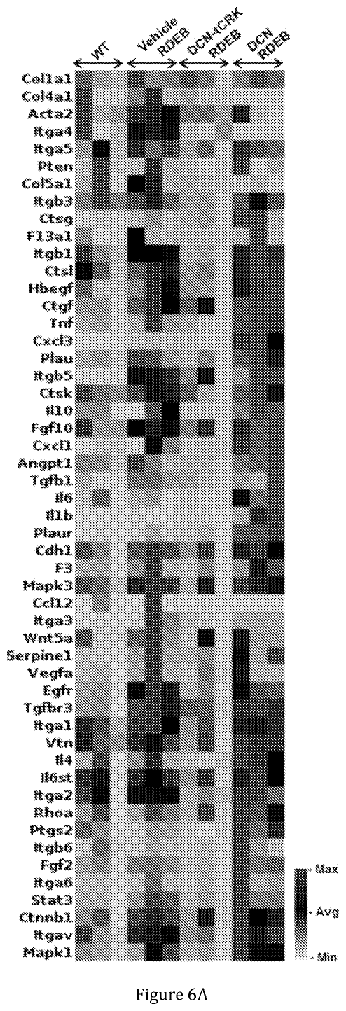

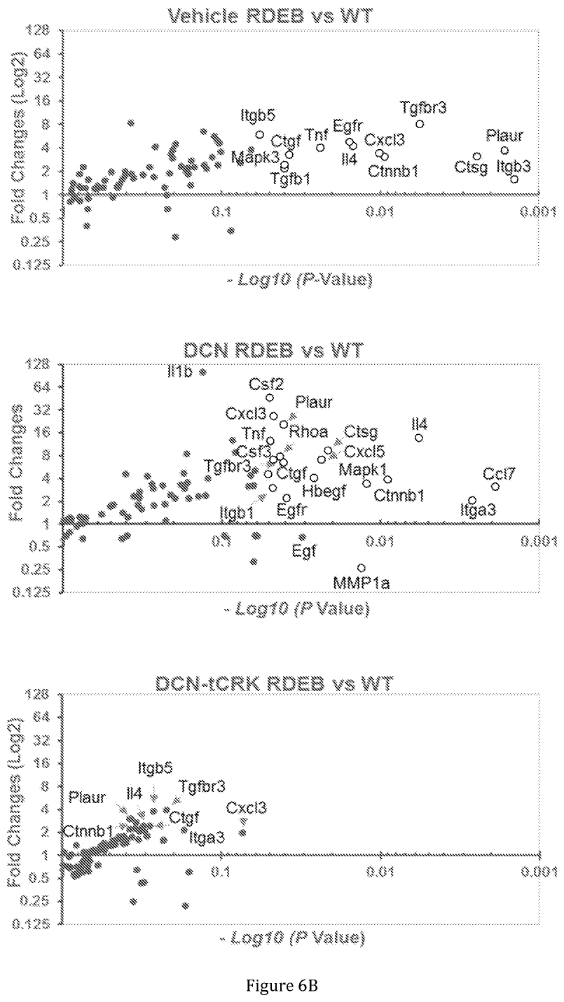

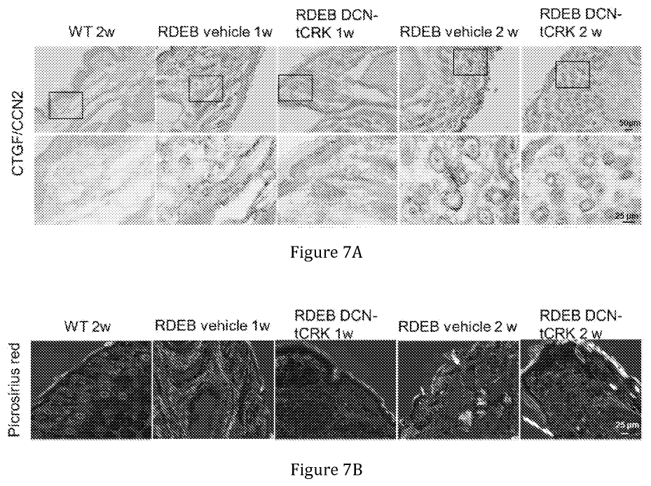

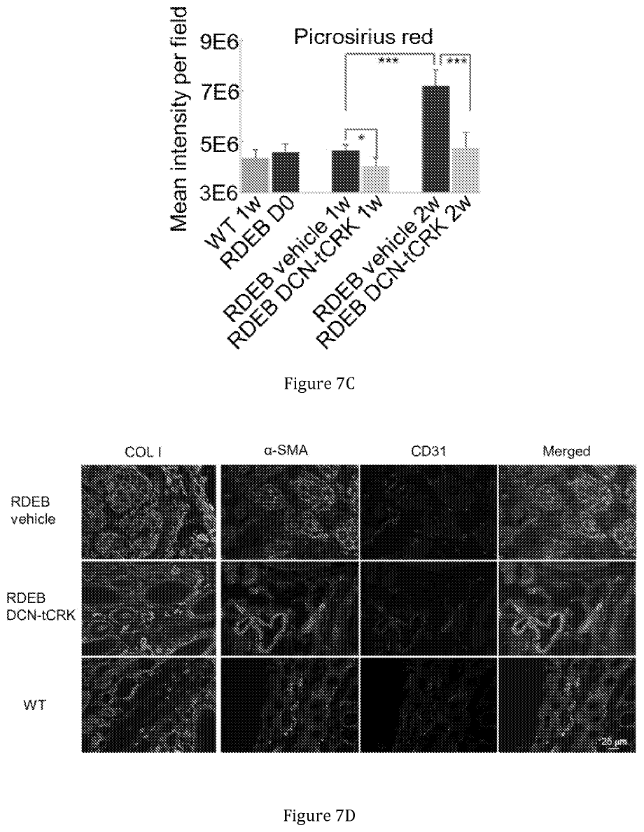

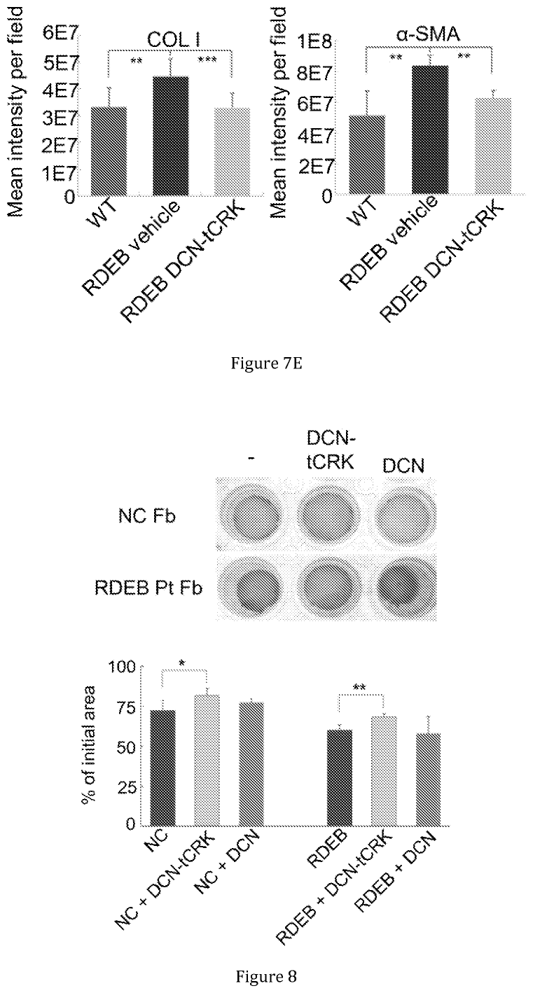

WT and mutant NRP1 were labeled with FAM and added to the immobilized plate. The binding of the NRP1 was measured based on fluorescent intensity. Error bars represent SEM. Experiments were repeated with triplicate samples **p<0.01, ***p<0.001, ****p<0.0001, Student's unpaired t-test. FIG. 1C shows the internalization of DCN-tCRK in the NRP-1 positive cells. FAM-labeled DCN-tCRK was incubated with PC3 and M21 cells positive and negative for NRP-1 expression respectively. DCN-tCRK was detected by anti-FAM immunostaining. Nuclei were counter stained with DAPI. Representative images from three independently studied experiment. Scale bar 20 μm.

FIGS. 2A to 2D illustrate recombinant protein production and characterization of an exemplary DCN-tCRK. FIG. 2A shows an example of a purification chromatogram after the HisTrap HP column step on the Äkta Start with one big peak, of which all peak fractions were used for further processing. In FIG. 2B Coomassie-stained reduced SDS-Page gel (upper panel) and Western blot (lower panel) of purified DCN-tCRK are shown alongside the DCN of prior art. On the SDS gel 2 and 1 μg of protein were loaded; for Western blot analysis 1 and 0.5 μg of protein were applied. Monomeric forms of the proteins, as well as forms including the GAG side chains are visible. FIG. 2C shows dynamic light scattering (DLS) measurements (n=3) on the hydrodynamic diameter of DCN-tCRK. FIG. 2D shows a differential scanning calorimetry (DSC) curve for the melting temperature of DCN-tCRK.

FIG. 3 illustrates the pharmacokinetics of DCN-tCRK compared to DCN. 5 mg/kg of DCN-tCRK or DCN was injected i.v. in healthy Balb/c mice. Blood samples were gathered and analyzed with standard ELISA for human DCN from eight time points. n=4 per group.

FIGS. 4A to 4D demonstrate that DCN-tCRK improves survival of col7a1^{-/-} mice and homes to the skin. FIG. 4A shows the Kaplan-Meier survival analysis of the col7a1^{-/-} mice that received DCN-tCRK (median life span: 11 days; n=21), DCN (median life span: 7 days; n=17) and PBS (median life span: 2 days; n=24) administration. FIG. 4B shows quantitation on the levels of DCN and DCN-tCRK, determined using Human Decorin ELISA kit, in the skin of recipient col7a1^{-/-} mice at one week, two weeks and three weeks post intrahepatic administration (n=3 per time points). There was no quantitation on the level of DCN at the three-week time point as no mice survived till that time after DCN administration. *p<0.05, **p<0.01 FIG. 4C shows immunohistochemical staining using anti-histidine antibody (anti-his) on both paw and dorsal skin of col7a1^{-/-} mice are presented. Nuclei were counterstained with DAPI. Scale bar: 20 μm. FIG. 4D shows representative double-staining of anti-histidine tag and anti-NRP-1 and the merged image (with DAPI counterstain) of the DCN-tCRK, DCN and untreated RDEB skin are presented. Scale bar: 25 μm

FIG. 5 shows the Kaplan-Meier survival analysis of the col7a1^{-/-} mice comparing the historical survival after dextran/human serum albumin (D/HSA; median life span: 3 days; n=29; historical data Liao et al. 2018, Stem Cell Transl Med, 7:530-542) administration with the survival after DCN-tCRK (median life span: 11 days; n=21) and DCN (median life span: 7 days; n=17) and PBS (median life span: 2 days; n=24) administration.

FIG. 6 illustrates that DCN-tCRK normalizes fibrotic gene signature in RDEB. FIG. 6A shows relative gene expression in a clustergram for the genes that had >1.5-fold increase in expression in the untreated RDEB skin as compared to the WT. FIG. 6B shows volcano plots on log 2 fold changes and −log 10 p value of gene expression in the vehicle, DCN and DCN-tCRK treated col7a1^{-/-} mouse skin relative to the WT.

FIG. 7 demonstrates that DCN-tCRK administration suppressed the development of fibrosis in col7a1^{-/-} mice. FIG. 7A shows representative immunohistochemical staining of CTGF/CCN2 in WT and col7a1^{-/-} mice at one and two weeks of age with and without DCN-tCRK treatment. Scale bar 50 μm upper panel and 25 μm lower panel. FIG. 7B shows picrosirius red staining of the paw skin from the WT and col7a1^{-/-} mice at one and two weeks of ages with and without DCN-tCRK treatment. Picrosirius red images were acquired using polarized light. Scale bar 25 μm. FIG. 7C shows quantification of the picrosirius red mean intensity per field acquired with a 20× objective. Eight fields or more were acquired per section and at least 4 sections were analyzed per biopsy. Scale bar 25 μm. *p<0.05, **p<0.01. FIG. 7D shows representative pictures of collagen type I (COL1) expression in RDEB and WT skin at two weeks (COL1 first column), and double immunofluorescence staining of α-smooth muscle actin (αSMA second column) and blood vessels (CD31 third column) in WT and col7a1^{-/-} mice at two weeks of age with and without DCN-tCRK treatment. Nuclei were counter-stained by DAPI. Merged image is shown in the fourth column. Scale bar 25 μm. FIG. 7E shows quantification of mean immunostaining intensity for COL1 and αSMA expression on skin sections (N=3 in each treatment group). *, ** and *** denote p≤0.05, 0.01 and 0.001 respectively.

FIG. 8 shows the results of an in vitro Collagen lattice contraction assay. Upper, representative images of human normal fibroblasts and RDEB patient-derived fibroblasts 48 hours after seeding in collagen gels, with and without addition of DCN and DCN-tCRK at a final concentration of 75 nM. Bottom, contraction of collagen gels, calculated as percentage of contraction compared with the initial area. Data (n=3) are presented as mean±SEM. *p<0.05, **p<0.001.

### DETAILED DESCRIPTION

It is to be understood that this invention is not limited to any particular methodology, protocols, reagents, and formulations described, as such may vary. It is also to be understood that the terminology used herein is for the purpose of describing particular embodiments only and is not intended to limit the scope of the present invention, which will be limited only by the appended claims.

As used herein, the singular expressions “a”, “an” and “the” mean one or more. Thus, a singular noun, unless otherwise specified, carries also the meaning of the corresponding plural noun.

The present invention relates to a therapeutic use of a homing peptide-guided decorin conjugate. More specifically, the invention provides a homing peptide-guided decorin conjugate for use in the treatment of epidermolysis bullosa, as well as a method of treating epidermolysis bullosa in a subject in need thereof by administering an efficient amount of a homing peptide-guided decorin conjugate to said subject.

Epidermolysis bullosa is a group of rare diseases that cause fragile, blistering skin. The blisters may appear in response to minor injury, even from heat, rubbing, scratching or adhesive tape. In severe cases, the blisters may occur inside the body, such as the lining of the mouth or the stomach. Epidermolysis bullosa exists in various forms, including acquired and congenital forms, the latter of which may be recessive or dominant. Non-limiting examples of epidermolysis bullosa include acquired epidermolysis bullosa, junctional epidermolysis bullosa, epidermolysis bullosa simplex, Kindler syndrome, and dystrophic epidermolysis bullosa, including dominant dystrophic epidermolysis bullosa and recessive dystrophic epidermolysis bullosa, such as recessive dystrophic epidermolysis bullosa inversa. Any subtypes of said examples are also encompassed.

As used herein, the term “subject” refers to an animal subject, preferably to a mammalian subject, more preferably to a human subject. Herein, the term “patient” refers to a human subject.

As used herein, the term “treatment” or “treating” refers to the administration of the conjugate or a pharmaceutical composition comprising the same to subject for purposes which may include ameliorating, lessening, inhibiting, or curing epidermolysis bullosa.

As used herein, the term “efficient amount” refers to an amount by which harmful effects of epidermolysis bullosa are, at a minimum, ameliorated.

As used herein, the term “decorin” (DCN) refers to any isoform of a small leucine-rich chondroitin sulfate proteoglycan. It is a multifunctional proteoglycan that, for example, regulates collagen fibril formation, prevents tissue fibrosis, promotes tissue regeneration, and acts as an antagonist of TGF-8. In some embodiments, decorin is human decorin comprising or consisting of an amino acid sequence of decorin isoform A, B, C, D or E, with or without N-terminal signal sequence and/or propeptide. In some embodiments, the decorin comprises or consists of an amino acid sequence set forth in any one of SEQ ID Nos: 6-20. Conservative sequence variants and peptidomimetics of said decorin species are also included. As used herein, the term “decorin segment” refers to a part of the present conjugate that comprises or consists of decorin.

In some embodiments, the decorin segment comprises or consists of an amino acid sequence that has at least about 99%, 98%, 97%, 96%, 95%, 90%, 80%, 70%, or 60% sequence identity with the amino acid sequence of SEQ ID NOs: 6-20, or any percentage in between, provided that the biological properties of decorin are not significantly altered. Such decorin variants may arise from addition, deletion and/or substitution of one or more amino acids. Means and methods for determining whether decorin has retained its biological properties are readily available in the art.

As used herein, the percentage of sequence identity between two sequences is a function of the number of identical positions shared by the sequences (i.e. % identity=# of identical positions/total # of positions×100), taking into account the number of gaps, and the length of each gap, which need to be introduced for optimal alignment of the two sequences. The comparison of sequences and determination of identity percentage between two sequences can be accomplished using mathematical algorithms available in the art.

As used herein, the term “homing peptide” refers broadly to any peptide that selectively homes to, i.e. targets, specific cells or tissue in vivo in preference to other cells or tissues. Accordingly, homing peptides can be utilized as targeted delivery vehicles.

The homing peptide-guided decorin conjugate for use in the present invention differs from the known decorin fusion protein disclosed in WO 2008/136869 at least in respect of the homing peptide employed. The prior art decorin fusion protein contains the known CRK peptide (CRKDKC; SEQ ID NO: 3), whereas the C-terminal end of the novel homing peptide utilized in the present invention consists of the amino acid sequence RKDK (SEQ ID NO: 1). In some embodiments, the C-terminal end of the novel homing utilized in the present invention consists of CRKDK (SEQ ID NO: 2).

It has now been surprisingly discovered that truncation of the C-terminal cysteine of the known CRK peptide (CRKDKC; SEQ ID NO: 3) changes the homing specificity of the peptide. While the CRK peptide homes selectively to skin wounds, the truncated CKR, denoted hereinafter as tCRK (RKDK, SEQ ID NO: 1; or CRKDK, SEQ ID NO: 2), confers the peptide the ability to home to and penetrate non-wounded skin while retaining its ability to home to skin wounds. In other words, the CRK peptide homes selectively to skin wounds only, whereas the tCRK peptide homes and penetrates selectively to both skin wounds and non-wounded skin.

The truncation of the C-terminal cysteine of the CRK peptide exposes a cryptic CendR (C-end Rule) sequence R/KXXR/K (SEQ ID NO: 4), i.e. RKDK (SEQ ID NO: 1) in the present tCRK peptide. Without being limited to any theory, the tCRK peptide can penetrate skin tissue through internalization by dermal microvascular endothelial cells that express NRP-1 on their cell surface. Interestingly, the CRK peptide containing the cryptic CendR-motif is not capable of penetrating cells and tissues (Järvinen and Ruoslahti, 2007, Am J Pathol 171: 702-711; Agemy et al., 2010, Blood 116: 2847-2856).

Accordingly, the homing peptide employed in the present conjugate comprises a tCRK element at the C-terminal end of the homing peptide.

As used herein, the term “C-terminal end” (also known as the carboxyl-terminus, carboxy-terminus, C-terminus, or COOH-terminus) refers to the end of an amino acid chain terminated by a free carboxyl group (—COOH). Herein, the terms “C-terminal end” and “C-terminal” are interchangeable.

As used herein, the term “N-terminal end” (also known as the amino-terminus, amine-terminus, N-terminus, or NH₂-terminus) refers to the start of an amino acid chain. The first amino acid of an amino acid chain contains a free amine group (—NH₂). Herein, the terms “N-terminal end” and “N-terminal” are interchangeable. Peptide sequences are written from N-terminus to C-terminus.

As used herein, the term “tCRK element” refers to a peptide having the amino acid sequence RKDK (SEQ ID NO: 1) or CRKDK (SEQ ID NO: 2) that selectively homes to skin and skin wounds in vivo, and can penetrate skin tissue. The terms “tCRK element” and “tCRK peptide” are interchangeable.

In accordance with the present invention, the tCRK element is located at the C-terminal end of the homing peptide employed herein. More specifically, the tCRK element is located at the C-terminal extremity of the homing peptide and comprises the terminal carboxyl group. In other words, the C-terminal end of the homing peptide consists of the amino acid sequence RKDK (SEQ ID NO: 1) or CRKDK (SEQ ID NO: 2). Thus, the homing peptide comprising the tCRK element ends with the amino acid sequence RKDK (SEQ ID NO: 1) or CRKDK (SEQ ID NO: 2).

In some embodiments, the homing peptide employed in the present invention consists of SEQ ID NO: 1 or SEQ ID NO: 2. In some other embodiments, the homing peptide comprises SEQ ID NO:1 or SEQ ID NO: 2. In the latter cases, the homing peptide comprises additional amino acids attached to the N-terminal end of the tCRK element. However, the C-terminal end of such longer homing peptides still consists of the tCRK element. In some embodiments, the homing peptide can comprise up to 100 amino acids. In some embodiments, the homing peptide can comprise up to 50 amino acids. In some embodiments, the homing peptide can comprise up to 20 amino acids. In some embodiments, the peptide homing can comprise up to 10 amino acids.

In some embodiments, the homing peptide may be part of a cyclic structure, and it may be cyclized, for example, via a disulfide bond, and then cleaved by a protease to expose the tCRK sequence as a CendR peptide in the C-terminus of the homing peptide.

As used herein, expression “tCRK-guided decorin” refers to any decorin conjugate, whose targeted delivery or homing is accomplished by the tCRK homing peptide according to any one of the embodiments disclosed herein. Non-limiting examples of such conjugates include those wherein the decorin segment comprises or consists of an amino acid sequence set forth in any one of SEQ ID NO: 6-20 and is attached from its C-terminal end to the N-terminal end of the tCRK element of SEQ ID NO: 1 or 2, with or without an intervening linker, such as that of SEQ ID NO: 23 or 24. Further examples include conjugates comprising or consisting of an amino acid sequence of SEQ ID NO: 21 or 22. Still further examples include sequence variants having at least about 99%, 98%, 97%, 96%, 95%, 90%, 80%, 70%, or 60% sequence identity to said sequences as well as their conservative sequence variants and peptidomimetics, with the proviso that the homing specificity and the penetration capability of the tCRK element, and the biological activity of decorin remains essentially unaltered.

In some embodiments, the tCRK-guided decorin conjugate may be provided for use in the form of a fusion protein but is not limited thereto. Accordingly, in some embodiments, the conjugate is a “fusion protein” comprising a decorin segment fused or linked to the N-terminal end of a homing peptide disclosed herein, preferably from the C-terminal end of the decorin segment, with or without one or more additional amino segments, such as peptide, oligopeptide, polypeptide or protein segments which may consist of or comprise natural or non-natural amino acids or peptidomimetics. Such one or more additional amino acid segments may be fused or linked to the N-terminal end of the decorin segment and/or fused or linked between the C-terminal end of the decorin segment and N-terminal end of the homing peptide. Said additional amino acid segments may have therapeutic activity, or they may be employed for diagnostic, imaging or visualization purposes, for example.

As used herein, the term “peptide” refers to a series of amino acid residues connected to one another typically by peptide (amide) bonds between the alpha-amino and carbonyl groups of the adjacent amino acids to form an amino acid sequence. Conventionally, peptides are defined as molecules that consist of between 2 and 100, e.g. between 2 and 50 amino acids. However, peptides may be subdivided into oligopeptides, which have few amino acids (e.g., 2 to 20), and polypeptides, which have many amino acids (e.g., 20 to 100, or 20 to 50). Proteins are essentially large peptides typically consisting of more than 50, or more than 100 amino acids. Thus, for the sake of simplicity of expression, the term “peptide” as used herein encompasses any peptide-bonded series of natural (L-) and/or non-natural (D-) amino acid residues, and is interchangeable with “oligopeptides”, “polypeptides”, “proteins” and fragments thereof, unless clearly indicated otherwise. Peptidomimetic forms of the peptides are also encompassed.

The fusion proteins for use in the present invention can have any suitable length, for example, up to 300, 350, 400, 500, 1000 or 2000 residues, or it may have any number of residues including or between said integers. As used herein, the term “residue” refers to an amino acid or amino acid analog.

In some embodiments, the fusion proteins for use in the present invention may comprise small peptide tags that facilitate, for example, purification, isolation, and/or detection. Non-limiting examples of suitable affinity tags for purification purposes include polyhistidine tags (His-tags), hemagglutinin tags (HA-tags), glutathione S-transferase tags (GST-tags), biotin tags, avidin tags and streptavidin tags. Suitable detection tags include, but are not limited to, fluorescent proteins, such as GFP.

Depending on their length, the fusion proteins for use in the present invention may be created by any appropriate means, methods or techniques available in the art, for example, by an automated peptide synthesizer, or produced by genetic engineering technologies. For example, an expression vector comprising a polynucleotide encoding for decorin and the tCRK homing peptide may be prepared by genetic engineering, and then transfected into a host cell to express the fusion protein. Non-limiting examples of suitable host cells include prokaryotic hosts such as bacteria (e.g. E. coli, bacilli), yeast (e.g. Pichia postoris, Saccharomyces cerevisae), and fungi (e.g. filamentous fungi), as well as eukaryotic hosts such as insect cells (e.g. Sf9), and mammalian cells (e.g. CHO cells, HEK cells). Expression vectors may be transfected into host cells by a wide variety of techniques commonly used for the introduction of exogenous DNA into a prokaryotic or eukaryotic host cell including, but not limited to, electroporation, nucleofection, sonoporation, magnetofection, heat shock, calcium-phosphate precipitation, DEAE-dextran transfection and the like. A wide variety of expression vectors are readily available in the art, and those skilled in the art can easily select suitable ones depending on different variables, such as the host cell to be employed. The fusion proteins for use in the present invention can also be produced by in vitro protein expression, also known as in vitro translation, cell-free protein expression, cell-free translation, or cell-free protein synthesis. Several cell-free expression systems based on, for instance, bacterial, rabbit reticulocyte, CHO, or human lysates are commercially available in the art. In vitro protein expression may be performed either in batch reactions or in a dialysis mode.

Fusion partners of the fusion protein for use in the present invention may be linked to each other directly or via a linker. The linker may be a peptide linker or a non-peptide linker. If the linker is a peptide linker, it may be composed of one or more amino acids. A non-limiting example of a peptide linker comprises or consists of an amino acid sequence set forth in SEQ ID NO: 23 or 24.

Furthermore, the homing peptide might be coupled to decorin or any other therapeutic protein comprised in the present conjugate or composition via a system like SpyTag/SpyCatcher.

In accordance with the above, the fusion proteins for use in the present invention may in some embodiments be produced using a nucleic acid molecule which encodes the fusion protein. Such nucleic acid molecules may be used not only for recombinant production of the fusion proteins they encode but also for gene therapy through means and methods available in the art.

Conservative sequence variants comprising of natural (L-) and/or non-natural (D-) amino acids and/or peptidomimetics of the fusion proteins are also envisaged for use in the treatment of epidermolysis bullosa.

The term “conservative sequence variant”, as used herein, refers to amino acid sequence modifications, which do not significantly alter the biological properties of the protein or peptide in question. Conservative sequence variants include variants arising from one or more amino acid substitutions with similar amino acids well known in the art (e.g. amino acids of similar size or with similar charge properties).

As used herein, the term “peptidomimetic” refers to a peptide-like molecule designed to mimic a given protein or peptide without altering its activity, such as homing specificity. Non-limiting examples of peptidomimetics include chemically modified peptides, D-peptide peptidomimetics, peptide-like molecules comprising non-naturally occurring amino acids, peptoids and β-peptides. Also molecules that resemble peptides, but which are not connected via a natural peptide linkage are included in the term. Means and methods for producing peptidomimetics are readily available in the art.

The tCRK-guided decorin conjugate for use in the treatment of epidermolysis bullosa may further comprise one or more covalently (directly or indirectly via a linker) or non-covalently linked additional moieties as desired, provided that the therapeutic activity of the conjugate is retained.

In some embodiments, an additional moiety may have therapeutic activity of its own, such as anti-inflammatory activity, anti-angiogenic activity, regenerative activity, pro-angiogenic activity, cytotoxic activity, pro-apoptotic activity, antimicrobial activity (e.g. anti-bacterial activity, anti-viral activity, anti-fungal activity or anti-protozoan activity), anti-fibrotic activity, anti-wrinkle activity, anti-itching activity, anti- or pro-transmitter (such as histamine) activity or cytokine activity, or it may be a cytokine inhibitor (e.g. an antagonist, a soluble receptor, a cytokine-binding molecule, or a cytokine that blocks other cytokines), to mention some non-limiting examples of potential biological activities or therapeutic effects of therapeutic moieties.

Accordingly, in some embodiments, an additional moiety may be a small molecule, such as that selected from anti-histamine, antibiotics, retinoids, benzoyl peroxide, podophyllotoxin, cytotoxic drugs, and immune modulators such as corticosteroid derivatives, calcineurin inhibitors and imiquimod. Furthermore, an additional moiety may be a protein moiety, such as anti-fibrotic TGF-β3, any regenerative or anti-inflammatory growth factor or cytokine such as interleukin-10 (IL-10), any angiogenic growth factor such as vascular endothelial growth factor (VEGF), any anti-apoptotic protein such as bit1, any inflammation suppressing enzyme such as CD73, or any collagen such as type VII collagen.

In some embodiments, an additional moiety may be employed to facilitate detection of the tCRK-guided decorin conjugate. Thus, the conjugate may comprise a detectable agent. As used herein, the term “detectable agent” refers to any molecule which can be detected, either directly or indirectly, preferably by a non-invasive and/or in vivo visualization technique. Non-limiting examples of detectable agents suitable for use in the disclosed conjugates include optical agents such as fluorescent agents including a variety of organic and/or inorganic small molecules and a variety of fluorescent proteins and derivatives thereof, phosphorescent agents, luminescent agents such as chemiluminescent agents, and chromogenic agents; radiolabels such as radionuclides that emit gamma rays, positrons, beta or alpha particles, or X-rays; non-radioactive isotopes such as cadolinium (Gd); ionic and non-ionic contrasting agents such as iodine-based contrasting agents; electromagnetic agents such as magnetic, ferromagnetic, paramagnetic, and/or superparamagnetic agents; upconverting nanoparticles (UCNP), resonance particles, quantum dots, and gold particles. Further suitable detectable agents are available in the art. Those skilled in the art can readily select an appropriate imaging technique depending on the type and species of the detectable agent employed in the conjugate. Such techniques include, but are not limited to, radiological techniques, isotope techniques such as positron emission tomography, ultrasound imaging and magnetic resonance imaging (MRI).

A detectable agent may be attached to the decorin conjugate directly, for example, through a covalent bond, or indirectly, for example, via a binding agent, a linker, or a chelating agent such as diethylenetriaminepentaacetic acid (DTPA), 4,7,10-tetraazacyclododecane-N—,N′,N″,N′″-tetraacetic acid (DOTA) and/or metallothionein.

Techniques for conjugating or otherwise associating a detectable agent to a peptide or protein conjugate are well known in the art. For example, conjugates comprising a detectable protein, such a fluorescent protein (e.g. GFP), can be produced as fusion proteins by recombinant techniques.

None of the disclosed homing peptide-guided decorin conjugates, more specifically tCRK-guided decorin conjugates, exists in nature.

In some embodiments, the tCRK-guided decorin conjugate for use in the treatment of epidermolysis bullosa is provided in a pharmaceutical composition comprising the conjugate and a pharmaceutically or physiologically acceptable carrier to enable administration in vivo.

As used herein, the term “pharmaceutical composition” refers broadly to a preparation of one or more of active ingredients and physiologically suitable components such as carriers, adjuvants and/or excipients. The purpose of a pharmaceutical composition is to facilitate administration of a compound to a subject or organism. As used herein, the term “active ingredient” refers broadly to a substance accountable for a biological effect including, but not limited to, anti-inflammatory effects, anti-angiogenic effects, regenerative effects, pro-angiogenic effects, cytotoxic effects, pro-apoptotic effects, antimicrobial effects (e.g. anti-bacterial effects, anti-viral effects, anti-fungal effects or anti-protozoan effects), anti-fibrotic effects, anti-itch effects, anti-transmitter effects, pro-transmitter effects (e.g. histamine), cytokine-induced effects or cytokine inhibition. In the context of the present disclosure, the term “active ingredient” refers particularly to the tCRK-guided decorin, although the composition and/or the conjugate may comprise further active agents as set forth above.

The pharmaceutical composition may be formulated as desired, for example as a semisolid or solid preparation, solution, dispersion, or suspension, using means and methods readily available in the art, for example by means of conventional mixing, dissolving, granulating, dragee-making, levigating, emulsifying, encapsulating, entrapping, lyophilizing or similar processes.

As used herein, the terms “pharmaceutically acceptable” and “physiologically acceptable” are interchangeable and refer to a material that is suitable for administration to a subject or organism without undue adverse side effects such as toxicity, significant irritation and/or allergic responses. In other words, the benefit/risk ratio must be reasonable.

As used herein, the term “pharmaceutically acceptable carrier” refers to a carrier substance or diluent with which the active ingredient is combined to facilitate administration and that is physiologically acceptable to the recipient. Pharmaceutically acceptable carriers are readily available in the art and, depending on the intended route of administration, may be selected from the group consisting of, but not limited to, transdermal carriers, transmucosal carriers, enteral carriers, parenteral carriers, and carriers for extended release formulations. The selected carrier should not abrogate the biological activity and properties of the active ingredient but minimize any degradation thereof as wells as minimize adverse side effects in the recipient.

As used herein the term “excipient” refers to a preferably inert substance added to a pharmaceutical composition to further facilitate administration of an active ingredient. Typical examples of different types of excipients, without limitation, include stabilizers, preservatives, pH modifiers, fillers, thickeners, viscosity modifiers, lubricants, solubilizers, surfactants, sweeteners, taste masking agents, and the like.

Useful stabilizing excipients include, but are not limited to, surfactants such as polysorbate 20, polysorbate 80 and poloxamer 407; polymers such as polyethylene glycols and povidones; carbohydrates such as sucrose, mannitol, glucose and lactose; sugar alcohols such as sorbitol, glycerol, propylene glycol and ethylene glycol; proteins such as albumin; amino acids such as glycine and glutamic acid; fatty acids such as ethanolamine; antioxidants such as ascorbic acid; chelating agents such as EDTA salts; and metal ions such as Ca, Ni, Mg and Mn. Among useful preservative agents, without limitation, are benzyl alcohol, chlorbutanol, benzalkonium chloride and possibly parabens. Among useful buffering excipients are, without limitation, sodium and potassium phosphates, citrate, acetate and carbonate or glycine buffers depending on the targeted pH-range. The use of sodium chloride as a tonicity adjuster is also useful. Non-limiting examples of further excipient materials include calcium carbonate, calcium phosphate, various sugars and types of starch, cellulose derivatives, gelatin, vegetable oils and polyethylene glycols. As readily understood by those skilled in the art, a given excipient may serve more than one function.

The pharmaceutical composition can be administered in a number of ways depending on whether local or systemic treatment is desired, and on the area to be treated. Administration can be for example parenteral, enteral or topical.

Parenteral administration of the composition, if used, is generally applied by injection, for example intravenously, intraperitoneally, subcutaneously, or intramuscularly. Preparations for parenteral administration are typically sterile aqueous or non-aqueous solutions, suspensions or emulsions, but the preparation may also be provided in a concentrated form or in a form of a powder to be reconstituted on demand. Slow release or sustained release formulation are also contemplated. Means and methods for formulating preparations for parenteral administration are readily available in the art, and those skilled in the art can easily select appropriate physiologically suitable carriers, adjuvants and/or excipients depending on the desired specifics of the preparation.

Non-limiting examples of aqueous carriers for parenteral and other pharmaceutical preparations include sterile water, water-alcohol solutions, saline, and buffered solutions at physiological pH. Parenteral vehicles include sodium chloride solution, Ringer's dextrose solution, dextrose plus sodium chloride solution, Ringer's solution containing lactose, or fixed oils. Intravenous vehicles include fluid and nutrient replenishers, electrolyte replenishers, such as those based on Ringer's dextrose solution, and the like.

Non-limiting examples of non-aqueous carriers for parenteral and other pharmaceutical preparations include solvents such as propylene glycol, polyethylene glycol, vegetable oils such as olive oil, fish oils, and injectable organic esters such as ethyl oleate.

If the parenteral preparation is provided as a concentrated solution or dispersion, or as a powder, aqueous or non-aqueous carriers mentioned above may be used for reconstitution. A solution for the reconstitution may be provided in the same package as the concentrate or powder. If lyophilization is used for preparing the powder, it may be beneficial to use cryoprotectants including, without limitation, polymers (e.g. povidones, polyethylene glycol, dextran), sugars (e.g. sucrose, glucose, lactose), amino acids (e.g. glycine, arginine, glutamic acid) and albumin.

Enteral administration of the composition, if used, may be applied, for example, through oral administration or administration via a percutaneous endoscopic gastrostomy (PEG). Compositions for oral administration include, without limitation powders, granules, capsules, sachets, tablets and aqueous or non-aqueous solutions and suspensions. Means and methods for formulating preparations for enteral administration are readily available in the art, and those skilled in the art can easily select appropriate physiologically suitable carriers, adjuvants and/or excipients depending on the desired specifics of the preparation.

Topical administration of the composition, if used, may be applied, for example, through transdermal administration, transmucosal administration, epicutaneous administration, intranasal administration, rectal administration, vaginal administration and administration by an inhalant. Depending on the administration route, formulations for topical administration can include ointments, lotions, creams, gels, drops, suppositories, sprays, liquids, powders and slow release or sustained release formulations or solid objects. Means and methods for formulating preparations for topical administration are readily available in the art, and those skilled in the art can easily select appropriate physiologically suitable carriers, adjuvants and/or excipients depending on the desired specifics of the preparation.

Some of the compositions can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Amounts and regimens for administration of a conjugate or a pharmaceutical composition disclosed herein can be determined readily by those with ordinary skill in the clinical art of treating skin diseases and conditions, especially epidermolysis bullosa. Generally, dosing will vary depending on considerations such as: age, gender and general health of the subject to be treated; kind of concurrent treatment, if any; frequency of treatment and nature of the effect desired; severity and type of epidermolysis bullosa in question; and other variables to be adjusted by the individual physician. A desired dose can be administered in one or more applications to obtain the desired results. For example, the pharmaceutical composition may be administered in a single daily dose, or the total daily dosage may be administered in divided doses of e.g. two, three or four times daily. The pharmaceutical composition may be provided, for example, in unit dosage forms or in extended release formulations.

### EXPERIMENTAL PART

Materials and Methods

Cloning of Decorin Fusion Proteins

Human decorin (DCN) cDNA (Krusius and Ruoslahti, 1986, PNAS 83:7683-787) without the native signal and pro-peptide sequence were cloned into the mammalian expression vector pEFIRES-P (Hobbs et al., 1998, Biochem Biophys Res Commun 252:368-372). The tCRK wound homing peptide cDNA was cloned to the C-terminus of decorin flanked by a stop-codon. A 6×His-tag was cloned to the N-terminus ahead of decorin. The construct was assembled by using the PIPE method (Klock and Lesley, 2009, Methods Mol Biol 498:91-103). For transformation NEB 5-alpha competent E. coli (high efficiency) cells were used (C2987H; New England Biolabs Ipswich, Mass.) according to the manufacturer's instructions. For plasmid purification (Mini-Prep), PCR purification and agarose gel purification, kits from Qiagen (Hilden, Germany) were used. DCN naturally forms a dimer (Scott et al., 2004, PNAS 101:15633-15638). The protein sequence of a monomeric 6×Histag-DCN-tCRK fusion protein is: G H H H H H H D E A S G I G P E V P D D R D F E P S L G P V C P F R C Q C H L R V V Q C S D L G L D K V P K D L P P D T T L L D L Q N N K I T E I K D G D F K N L K N L H A L I L V N N K I S K V S P G A F T P L V K L E R L Y L S K N Q L K E L P E K Met P K T L Q E L R A H E N E I T K V R K V T F N G L N Q Met I V I E L G T N P L K S S G I E N G A F Q G Met K K L S Y I R I A D T N I T S I P Q G L P P S L T E L H L D G N K I S R V D A A S L K G L N N L A K L G L S F N S I S A V D N G S L A N T P H L R E L H L D N N K L T R V P G G L A E H K Y I Q V V Y L H N N N I S V V G S S D F C P P G H N T K K A S Y S G V S L F S N P V Q Y W E I Q P S T F R C V Y V R S A I Q L G N Y K G S E F C R K D K Stop (SEQ ID NO: 21).

A schematic map of the DCN-tCRK fusion protein used in the experimental part is shown in FIG. 1A. As clearly set forth in the detailed description, the DCN-tCRK fusion protein of FIG. 1A is a non-limiting example of tCRK-guided decorin conjugates suitable for use in the present invention.

Recombinant Protein Production

The constructs in pEFIRES-P expression vector were transfected via lipofection (FuGene 6, Promega, Madison, Wis.) into HEK293F cells. Positive clones were selected in the culture medium composed of DMEM Hi-glucose (4.5 g/l)+2 mM L-alanyl-L-glutamine, 100 IU/ml penicillin (all from Sigma Aldrich, St. Louis, Mo.), and 10% FBS (Gibco, Grand Island, N.Y.), in the presence of 5-160 μg/ml puromycin (HyClone, Thermo Fisher Scientific). Established cell lines were maintained in the culture containing 10 μg/ml puromycin.

The validated cells were then resuspended in serum-free OptiCHO medium (Gibco) supplemented with 2 mM L-alanyl-L-glutamine (Sigma) and cultured in square shaped glass bottles mounted on a rotating shaker at in 37° C. in a 5% CO₂ atmosphere. After the cells reached a density of 1-2×10⁶ cells/ml, they were cultured further for 4 d at 33° C. for recombinant protein expression and secretion to the culture media. The protein was purified from the culture media via two step HisTrap purification protocol on the Äkta Start chromatography system (GE Healthcare, Munich, Germany)

Recombinant Protein Purification

Cell culture supernatants were filtered and degassed on ice through a 0.45 μm filter unit (Corning #430514, Corning, N.Y.). The 6×His-tagged proteins were purified by Ni-NTA-IMAC via a two-step purification protocol using first a HisTrap Excel column followed by a HisTrap HP column on the Äkta Start chromatography system (GE Healthcare, Munich, Germany) according to the manufacturer's instructions in a 4° C. cold cabinet. Buffers were prepared from the His Buffer Kit (GE Healthcare/VWR (11-0034-00). All buffers were filtered and degassed.

The HisTrap Excel column eluate was diluted in 20 mM sodium phosphate buffer (pH 7.4) with 0.5 M NaCl to a final imidazole concentration of 30 mM, and then further purified on a HisTrap HP column, with a 35 mM imidazole wash and a gradient elution up to 300 mM imidazole (FIG. 2A includes an example of such a purification chromatogram). The peak fractions were analyzed on a SDS NuPAGE 4-12% gradient gel (Life Technologies/Thermo Fisher Scientific, Waltham, Mass.) and visualized via PageBlue Protein Staining Solution (Thermo Fisher Scientific, Waltham, Mass.).

Selected peak fractions were pooled and dialyzed against cold TBS buffer (pH 7.6) using 50 kDa MWCO Float-A-Lyzers (Fisher Scientific/Spectrum Labs), before concentration via 10 kDa MWCO VivaSpin 6 tubes (GE Healthcare). Samples were filter sterilized (Ultrafree-MC GV Centrifugal Filter 0.22 μm, Millipore, Burlington, Mass.) and the protein concentration measured at A280 nm via Nanodrop (Thermo Fisher Scientific, Waltham, Mass.). All steps were performed at 4° C. or on ice. Sterile Tween-20 was added to a final concentration of 0.05% to prevent aggregation, before freezing aliquots rapidly at −80° C.

Recombinant protein was verified by SDS Page and Western blotting. BioRad's wet tank Mini-PROTEAN Trans-Blot Cell system was used (according to the manufacturer's instructions). A PVDF membrane was probed with a primary murine antibody against human decorin (MAB143, R&D Systems, Minneapolis, Minn.) according to the manufacturer's protocol. A secondary horseradish peroxidase-coupled anti-mouse antibody from Cell Signaling Technology was used. Chemiluminescent blot images were captured via ImageQuant LAS 4000 mini (GE Healthcare).

Biophysical Protein Analysis

The hydrodynamic diameter was measured by Dynamic Light Scattering (DLS) using a Zetasizer Nano ZS instrument (Malvern Instruments Ltd, Worchestershire, UK). The DCN-tCRK protein sample was diluted 1:5 in TBS buffer. Three 10×10 s measurements were performed at 25° C. Data were analyzed using the Zetasizer software v7.11 (Malvern Instruments Ltd.) via the protein analysis model (non-negative least squares analysis followed by L-cuve) and size distribution by volume.

The unfolding temperature of DCN-tCRK was determined using the VP-Capillary DSC (differential scanning calorimetry) instrument (GE Healthcare, Microcal Inc./Malvern Instruments Ltd.) in TBS buffer (50 mM Tris-Cl, 150 mM NaCl, pH 7.5) with a protein concentration of 0.2 mg/ml. All solutions were degassed. Samples were heated from 20° C. to 130° C. at a scanning rate of 2° C./min. Feedback mode was set to low′ and the filter period was 5 s. The melting temperature Tm (transition midpoint) was calculated by a Non-2-state fitting model using Origin 7.0 DSC software suite (Microcal Inc.).

Expressed recombinant DCN-tCRK protein was identified from the monomeric gel band using Eksigent 425 NanoLC coupled with Sciex high speed TripleTOF™ 5600+ mass spectrometer. After isolation of gel band and Coomassie stain removal protein was then subjected to reduction (TCEP, 25 mM), alkylation (iodoacetamide, 0.5 M), and trypsin digestion as described in detail in Vähätupa et. al., 2018. After trypsin digestion peptides were diluted to 14 μl of sample buffer (2% acetonitrile, 0.1% formic acid) and 1 μl of sample was injected to the triple TOF mass spectrometry.

In Vitro Binding Analyses

In vitro binding of DCN-tCRK and peptides to NRP-1 was analyzed using ELISA analysis. 96-well, black FLUOTRAC™ 600, high binding plates (Greiner Bio-One, Kremsmünster, Austria) were coated with 1004/well of 100 μg/ml DCN-tCRK in PBS at 4° C. overnight. 10 μg/well RPARPAR (SEQ ID NO: 25) and RPAPRARA (SEQ ID NO: 26) peptides were coated in parallel as a positive and negative control, respectively. BSA was used as an immobilization control. The plates were washed 3 times with phosphate buffered saline (PBS) and blocked for 1 h at 37° C. with 300 μl of blocking solution (1×PBS, 1% BSA, 0.1% Tween-20). His-tagged neuropilin-1 b1b2 domain (NRP-1 WT) and triple mutant NS346A-E348A-T349A neuropilin-1 b1b2 domain (NRP-1 mutant) were expressed and purified at the Protein Production and Analysis Facility at the Sanford Burnham Prebys Medical Discovery Institute (La Jolla, Calif.) as described previously (Teesalu et al., 2009, PNAS 106:16157-16162). The recombinant proteins NRP1 WT, NRP1 mutant, and DCN-tCRK were FAM (5-(and-6)-Carboxyfluorescein, #90024, Biotium Inc, CA, USA) labeled by mixing 1:10 ratio of amine-reactive FAM dye (diluted in DMSO final concentration 0.2%) and protein. The mixture reaction was incubated in the dark for 2 hours at RT, followed by ultrafiltration/dialysis with PBS to separate free dye from the protein. 100 μl of FAM-labeled NRP1 WT or NRP1 mutant protein in blocking solution was added to each well (20 μg/well), incubated at room temperature for 4-6 hours at room temperature or 4° C. overnight, and washed 3 times with blocking solution. After adding 100 μl PBS in each well, the plate was immediately read with top read mode using a fluorescence reader (Flex Station II, Molecular Devices; peak excitation=485 nm, peak emission=530 nm, cut off=515).

For the binding of FAM-DCN-tCRK to NRP-1 positive prostate carcinoma-3 (PC-3) cells (gift from the Ruoslahti laboratory at Sanford-Burnham-Prebys Medical Discovery Institute, La Jolla, Calif.) and negative melanoma (M21) cells (gift from David Cheresh Lab at University of California San Diego, La Jolla, Calif.) in vitro, the cells were first cultured in growth medium composed of 10% fetal bovine serum (FBS) in DMEM high glucose medium supplemented with penicillin, and streptomycin (Gibco). For experiments, the medium was aspirated, the cells were washed twice with warm medium, and fresh medium was added along with 10 μg FAM-labeled DCN-tCRK recombinant protein. The labelling was done by directly coupling DCN-tCRK recombinant protein to Fluorescein using Lightning-Link Fluorescein kit (Expedon Ltd, UK) according to the manufacturer protocol. The cells were incubated at 37° C. for one hour; medium was aspirated, the cells were washed and fixed with −20° C. methanol. The cells were washed with PBS and blocked (PBS, 1% BSA, 1% FBS, 1% goat serum, 0.05% Tween-20) for 30 minutes at RT followed by primary anti-FITC (Invitrogen, CA, USA. Catalog #A-889) for one hour at RT. The cells were washed, and secondary antibodies Alexa Fluor 488 goat anti-rabbit IgG (Invitrogen, USA) were applied for one hour at RT in the dark. The nuclei of cells were stained with DAPI. The coverslips were mounted on glass slides with Fluoromount-G (Electron Microscopy Sciences, PA, USA), imaged using confocal microscopy (Olympus FV1200MPE, Tokyo, Japan) and analyzed using the FV10-ASW4.2 viewer.

Mice and Study Approvals

BALB/cJRj mice (Janvier Labs, Le-Genest-Saint-Isle, France) were used in pharmacokinetics. The mice were fed with standard laboratory pellets and water ad libitum. All animal experiments with the Balb/cJRj mice were performed in accordance with protocols approved by the National Animal Ethics Committee of Finland (ESAVI/6422/04.10.07/2017).

An animal model of recessive dystrophic epidermolysis bullosa (RDEB), namely the col7a1−/− RDEB mice, was used to study the skin homing and therapeutic function of DCN-tCRK. The col7a1−/− RDEB mice were generated by breeding C57BL6/J col7a1+/− mice with the genotype determined by polymerase chain reaction (PCR). C57BL6/J col7a1+/− mice, kindly provided by Dr. Jouni Uitto at Thomas Jefferson University, were developed by targeted ablation of the col7a1 gene through out-of-frame deletion. All animal studies with the col7a1^{-/-} RDEB were conducted using protocols approved by New York Medical College Institutional Animal Care & Use Committee (IACUC).

Recombinant Protein Pharmacokinetics

Recombinant proteins DCN-tCRK or DCN were diluted in Tris buffered saline (TBS) containing 0.05% Tween-20. The pharmacokinetics of DCN-tCRK and DCN were studied with 8 week old Balb/c male mice. 5 mg/kg either DCN-tCRK or DCN was injected in tail vein under isoflurane anesthesia. Blood samples from distinct tail vein were gathered at 15 min, 30 min, 60 min, 2 h, 4 h, and 16 h after injection. At 8 h or 24 h after the injection, the mice were sacrificed under medetomidine-ketamine anesthesia and blood samples were collected from the subclavian vein. The samples were mixed with 1 M ethylenediaminetetraacetic acid (EDTA), centrifuged 2000 g for 10 min at room temperature and the plasma was stored for analysis. The concentration of human origin decorin in the plasma samples was determined with Human Decorin DuoSet ELISA kit (#DY143, R&D Systems) according to instructions provided by the manufactured. A venous blood sample from an uninjected mouse was used in each plate to ensure the specificity of the primary antibody.

Administration of DCN-tCRK and DCN in col7a1^{-/-} Mice

The pregnant col7a1^{+/-} mice were housed individually and monitored daily before delivery. As intravenous injection in neonatal mice is technically challenging and often yields inconsistent results, the Inventors chose to inject within 24 hours of birth the first dose of DCN-tCRK and DCN (5 μg in 15 μl PBS, corresponding to ˜5 mg/kg) into the liver of the col7a1^{-/-} mice, since liver is a primary site of hematopoiesis in fetal and neonatal mice and the human cells have been shown to rapidly enter the circulation after intrahepatic injection (Liao et al., 2015, Stem Cells 33:1807-1817; Liao et al., 2018, Steml Cells Transl Med 7:530-542). This first dose was followed by repeated intraperitoneal (i.p.) administration of the protein every other day till the mice reached 14 days of age (maximal 7 doses) and the dose was increased to 10 μg when the mice became a week old. The mice were monitored every day. All the experimental col7a1^{-/-} mice were genotyped at the time of sample collection.

Histological and Immunohistochemical Staining and hDCN Quantitation in col7a1^{-/-} Mice

Dorsal skin and paws (front and rear) were excised from selected mice, embedded in Tissue-Tec OCT Compound (Sakura Finetek, Torrance, Calif.) and stored at −80° C. freezer. 6 μm serial sections were cut for each specimen. Picrosirius Red staining and CTGF (#ab6992, Abcam, Cambridge, UK) immunohistochemical staining were performed at the Core Histology Lab of New York Medical College. For immunochemical staining of his tag, the sections were fixed in 4% paraformaldehyde and blocked with M.O.M. blocking reagent (Vector Laboratories, Burlingame, Calif.) (for antibodies raised in mouse) (Vector Laboratories, Burlingame, Calif.) or 10% horse serum (GIBCO, Grand Island, N.Y.) with 0.1% Triton (Sigma, St. Louis, Mo.). The slides were then incubated with respective primary antibodies, including anti-Col1A (#R1038, Acris, Rockville, Md.), anti-αSMA (#14968, Cell signaling Technology, Danvers, Mass.), anti-6×-His tag (#R930-25, Thermofisher Scientific, Carlsbad, Calif.) and anti-NRP-1 (#AF566-SP, R&D Systems, Minneapolis, Minn.) followed by corresponding Alexa Fluor 488 secondary antibodies (Invitrogen, Carlsbad, Calif.). The slides were then mounted in Vectashield mounting medium containing DAPI (Vector Laboratories, Burlingame, Calif.). Images were acquired using Nikon 90i Eclipse microscope (Nikon Instrument Inc., NY) using the same settings between the different groups in each set of experiments. Intensity of the immunostaining per field was measured using NIS-Elements AR software, following the user's guide. The RGB images were used for the quantitation of picrosirius red staining and the threshold was defined by choosing reference points within the image.

The homing of DCN-tCRK and DCN to skin in col7a1^{-/-} mice was determined using Human Decorin DuoSet ELISA kit (#DY143, R&D Systems, Minneapolis, Minn.) according to the manufacture's recommendations. Tissue biopsies were snap frozen in liquid nitrogen, ground with a precooled pestle, and homogenized with lysis buffer (1% Tween 20, protease inhibitor cocktail, DNase and RNase in PBS). After centrifugation at 12,000 g for 10 min at 4° C., the supernatant was collected and quantitated for total protein concentration with the BioRad DC protein assay (BioRad, Hercule, Calif.). Sera from col7a1^{-/-} mice with and without DCN-tCRK or DCN administration were diluted 1:20 in sample diluent before applying to the assay.

RT² Profiler PCR Wound Healing Pathway Analysis

The expressions of genes involving in mouse wound healing pathway were studied using RT² Profiler PCR Array (QIAGEN, Hilden, Germany). RT² Profiler Array contains primers for 84 wound-healing genes and 5 housekeeping genes with genomic DNA, reverse-transcriptional and PCR positive controls in 96 well plate. Total RNA was isolated from whole front paw of WT, RDEB and DCN or DCN-tCRK injected col7a1^{-/-} mice (3 mice in each group) at day 7. Quality and concentration of RNA was determined with NanoDrop 200C (ThermoScientific, Waltham, Mass.). RNA was treated with genomic DNA elimination mix (QIAGEN). 500 ng of total RNA of each sample was applied for reverse transcription using RT² First Strand kit (QIAGEN). cDNA synthesis reaction was combined with 2×RT² SYBR Green Master mix and 25 μl of this cocktail was dispensed in each well of 96-well plate. Q-PCR was run on QuantStudio5 Real-Time PCR instrument (Applied Biosystems, Foster City, Calif.). CT values were exported to an Excel file. Resulting raw data was analyzed using the PCR Array Data Analysis Template in the GeneGlobe Data Analysis Center (https://www.qiagen.com/us/geneglobe). A gene expression was calculated using the ΔΔC_{T} method. A fold-change gene expression threshold of 1.5 and a p-value threshold of 0.05 were used to analyzed data between WT pup and untreated/treated pups.

Collagen Lattice Contraction Assay

Human normal fibroblasts and RDEB patient-derived fibroblasts were cultured in DMEM supplemented with 10% FBS, as previously described (Liao et al., 2018, Stem Cells 36:1839-1850). The collagen lattices were prepared by mixing the cell suspension with neutralized rat tail collage type I (Advance BioMatrix, Carlsbad, Calif.). The final concentration of collagen was 2.4 mg/ml with a cell density of 2.1×10⁵ cells/ml. 500 μl of cells/collagen suspension was dispensed into a single well of 24-well plate and allowed to solidify for 30 min at room temperature. 0.5 ml of DMEM supplemented with 5% of FBS was added in each well after collagen polymerization and plates were cultured at 37° C. with 5% CO₂. After 12 hours of incubation, the gel from each well was gently released by the thin pipet tip and DCN or DCN-CRK were added respectively at a final concentration 75 μM (n=3 per condition). Images were acquired at 12 hours (initial area) and 48 hours (contraction area) respectively and the areas of gels were quantitated using Image J.

Statistics

Kaplan-Meier analysis was applied to determine the median life span and log-rank (Mantel-Cox) test was used to compare survival between different experimental groups (GraphPad Prism 6). Student's unpaired t-test was used to study DCN-tCRK binding to NRP-1. P values under 0.05 were considered significant.

Results

Generation of Multi-Functional, Recombinant DCN-tCRK Fusion Protein

The Inventors engineered DCN-tCRK fusion protein by placing tCRK peptide at the C-terminus of DCN (FIG. 1A). Both DCN-tCRK and native DCN were expressed in mammalian cells and purified using chromatography (FIG. 2A). Both recombinant proteins migrated as sharp bands at about 55 kDa with a smear above the band in SDS gel electrophoresis and detected as DCN by Western blot analysis (FIG. 2B). The sharp band corresponds to the core protein, and the smear is caused by heterogeneity in the glycosaminoglycan sulfate chain (mostly chondroitin) attached to the DCN core. Mass spectrometry validated the identity of DCN and the C-terminal tCRK sequence (Table 1). The hydrodynamic size indicates that DCN-tCRK exists as homogenous and non-aggregated macromolecules with a diameter consistent with the reported dimer of DCN (Scott et al., 2003, J Biol Chem 278:18353) (FIG. 2C). Differential scanning calorimetry produced a sharp peak with a melting temperature (Tm) of 49° C., suggesting that tCRK-DCN will maintain a stable tertiary structure at a physiological condition (FIG. 2D).

TABLE 1
The sequence of human DCN and the tCRK sequence
in the C-terminus analyzed by mass spectrometry.

| | | | % | | | Peptides |
|---|---|---|---|---|---|---|
| N | Unused | Total | Cov | Accession # | Name | (95%) |
| 1 | 54, | 54, | 45, | sp\|P07585\| | Decorin OS = Homo sapiens | 48 |
| | 45 | 45 | 7 | PGS2_HUMAN | GN = DCN PE = 1 SV = 1 | |
| | | | | | MKATIILLLLAQVSWAGPFQQRGLFDFML | |
| | | | | | EDEASGIGPEVPDDRDFEPSLGPVCPFRCQ | |
| | | | | | CHLRVVQCSDLGLDKVPKDLPPDTTLLDL | |
| | | | | | QNNKITEIKDGDFKNLKNLHALILVNNKIS | |
| | | | | | KVSPGAFTPLVKLERLYLSKNQLKELPEKM | |
| | | | | | PKTLQELRAHENEITKVRKVTFNGLNQMI | |
| | | | | | VIELGTNPLKSSGIENGAFQGMKKLSYIRIA | |
| | | | | | DTNITSIPQGLPPSLTELHLDGNKISRVDAA | |
| | | | | | SLKGLNNLAKLGLSFNSISAVDNGSLANTP | |
| | | | | | HLRELHLDNNKLTRVPGGLAEHKYIQVVY | |
| | | | | | LHNNNISVVGSSDFCPPGHNTKKASYSGVS | |
| | | | | | LFSNPVQYWEIQPSTFRCVYVRSAIQLGNY | |
| | | | | | K (SEQ ID NO: 6) | |
| 1 | 66 | 66 | 46, | sp\|P07585\| | Decorin OS = Homo sapiens | 63 |
| | | | 7 | PGS2_HUMAN | OX = 9606 GN = DCN PE = 1 SV = 1 | |
| | | | | | MKATIILLLLAQVSWAGPFQQRGLFDFML | |
| | | | | | EDEASGIGPEVPDDRDFEPSLGPVCPFRCQ | |
| | | | | | CHLRVVQCSDLGLDKVPKDLPPDTTLLDL | |
| | | | | | QNNKITEIKDGDFKNLKNLHALILVNNKIS | |
| | | | | | KVSPGAFTPLVKLERLYLSKNQLKELPEKM | |
| | | | | | PKTLQELRAHENEITKVRKVTFNGLNQMI | |
| | | | | | VIELGTNPLKSSGIENGAFQGMKKLSYIRIA | |
| | | | | | DTNITSIPQGLPPSLTELHLDGNKISRVDAA | |
| | | | | | SLKGLNNLAKLGLSFNSISAVDNGSLANTP | |
| | | | | | HLRELHLDNNKLTRVPGGLAEHKYIQVVY | |
| | | | | | LHNNNISVVGSSDFCPPGHNTKKASYSGVS | |
| | | | | | LFSNPVQYWEIQPSTFRCVYVRSAIQLGNY | |
| | | | | | KGSEF [symbol] (SEQ ID NO: 22) | |

The underlined letters indicate the peptides that were found to be specific to human DCN and the letters in italics indicate amino acids specific for the C-terminus including the tCRK sequence (CRKDK/RKDK), which is further indicated in bold.

DCN-tCRK Interacts with NRP-1 In Vitro

The Inventors next investigated whether the tCRK peptide fused to DCN retains its ability to interact with NRP-1. DCN-tCRK was immobilized on ELISA plates and tested its binding to wild type (WT) or mutant NRP-1, where the CendR-binding pocket was disabled by a triple mutation. (Teesalu et al., 2009, PNAS 106:16157-16162) DCN-tCRK effectively binds to WT NRP-1 at a significantly higher level than the control bovine serum albumin (BSA) (p<0.01), whereas it showed no significant binding to the mutant NRP-1 (p>0.05) (FIG. 1B). Furthermore, parallel studies with a synthetic RPARPAR (SEQ ID NO: 25) peptide, a prototypic CendR peptide, and RPARPARA (SEQ ID NO: 26), a control peptide with a C-terminally capped CendR-sequence and unable to interact with NRP-1, were used to fortify that the binding is dependent on CendR-sequence (FIG. 1B). The Inventors further determined whether DCN-tCRK binds to the cells that express NRP-1, i.e., human PC3 prostate carcinoma cells. M21 melanoma cells that do not express NRP-1 were also included in the assay. Supporting the NRP-1 dependent cell binding and penetration properties, internalization of DCN-tCRK was observed only in the NRP-1 positive PC3 cells, but not in the NRP-1 negative M21 cells (FIG. 1C).

DCN-tCRK and DCN Exhibited Similar Pharmacokinetics In Vivo

To determine whether the addition of tCRK peptide had any effect on the circulation half-life of DCN, DCN-tCRK and DCN were injected intravenously in parallel in healthy Balb/c mice and their amount in peripheral blood at different time points within 24 hours of administration was quantitated by ELISA. The half-life of DCN-tCRK in blood was 30 minutes and was not significantly different from that of DCN (FIG. 3). The pharmacokinetic studies suggest that modification of DCN with small vascular homing peptide does not influence the pharmacokinetics of DCN.

DCN-tCRK Administration Improves the Survival of col7a1^{-/-} Mice

Therapeutic function and skin homing properties of DCN and DCN-tCRK were evaluated in col7a1^{-/-} mice, an animal model of RDEB. These mice are generated by breeding of the heterozygous littermates, and col7a1^{-/-} mice can be identified at birth based on manifestation of hemorrhagic blistering in the skin. The newborn col7a1^{-/-} mice were randomly divided to receive DCN, DCN-tCRK or PBS (negative control) intrahepatic administration. Repeated intraperitoneal administration was performed to the surviving mice within each group every other day after the first dose until day 14. Here, the median life span of col7a1^{-/-} mice was 2 days after PBS injection and it was significantly prolonged to 7 days after administration of DCN (p<0.0001) (FIG. 4A). However, the survival of col7a1^{-/-} mice after DCN administration was not statistically significant as compared to a historical administration of dextran/human serum albumin (D/HSA), which was used as the vehicle for stem cell administration and sporadically increased the survival of some col7a1^{-/-} recipient mice likely by adjusting fluid balance (FIG. 5). Moreover, DCN injections did not extend the survival of the recipients beyond two weeks of age. Importantly, the median life span of the mice after DCN-tCRK treatment was further extended to 11 days, which was significantly better than that after PBS (p<0.0001) or historical D/HSA administration (p<0.001) (FIGS. 4A and 5). In addition, 85% of DCN-tCRK treated mice reached 7 days of survival and 20% of these mice survived past three weeks of age and were subsequently sacrificed for skin analyses.

DCN-tCRK Homes in Skin of col7a1^{-/-} Mice

An ELISA assay was utilized to quantitate human DCN and DCN-tCRK in the skin of recipient RDEB mice at one, two and three weeks (n=3 all time-points) (FIG. 4B). There was no statistically significant difference between DCN-tCRK and DCN treated skin at the one-week time point. However, the level of DCN-tCRK at the two-week time point was significantly higher than that of DCN (3.6-fold, p<0.05) (FIG. 4B). In addition, as the last i.p. administration of DCN-tCRK was conducted on day 14, identification of DCN-tCRK in the three-week skin (19.47±12.80 pg/ml) is highly suggestive of its stability in vivo for at least 7 days.

Immunohistochemical staining based on the expression of histidine-tag was also performed to analyze the anatomical distribution of DCN-tCRK or DCN in the RDEB skin. DCN-tCRK was detected in the dermis of both the paw and dorsal skin of the RDEB mice at one, two and three weeks (FIG. 4C). Moreover, staining of the gastrointestinal (GI) tract of the recipient RDEB mice did not reveal reactivity with anti-his antibody (data not shown), suggestive of a skin-specific targeting of DCN-tCRK. In contrast, although ELISA demonstrated the presence of DCN in the skin lysate, the anti-his immunostaining on DCN treated RDEB skin, represented by the one-week time point, only appeared to be non-specific (diffuse) (FIG. 4C). Further supporting the Inventors' non-limiting hypothesis that the homing of DCN-tCRK is afforded by NRP-1 dependent cell and tissue penetration, the anti-his and -NRP-1 double staining demonstrated that the signal from DCN-tCRK was within or in a close proximity to the cells that were positive for NRP-1 in RDEB skin (FIG. 4D)

DCN-tCRK Therapy Suppresses the Fibrotic Responses in RDEB Mice

Recent studies by the present Inventors have demonstrated a significant elevation of TGFβ signaling in col7a1^{-/-} mice beginning in the interdigital folds of the paws as early as a week after birth. Therefore, in this study, the skin biopsies of this time point were chosen for comparison of the expression of 84 genes central to wound healing responses and fibrosis formation between the WT and vehicle (D/HSA), DCN or DCN-tCRK-treated RDEB skin (n=3 per group) (Table 2). Relative to the WT, more than half of the genes showed >1.5-fold increase in expression in the vehicle-injected RDEB skin, as demonstrated in the clustergram in FIG. 6A. The relative fold changes (log 2) of gene expression and the p values (−log 10) are also presented as volcano plots and the significantly (p<0.05) dysregulated genes are marked in white in each plot (FIG. 6B). The significantly upregulated genes in the vehicle RDEB skin are involved in TGF signaling (i.e., Tgfb1, Tgfbr3, Ctgf), WNT signaling (Ctnnb1), MAPK1/MAPK3 signaling (Mapk3) and epidermal growth factor receptor signaling (Egfr), ECM remodeling (Ctsg, Plaur), cell adhesion (Itgb3, Itgb5) and inflammation (Il4, Cxcl3, Tnfα). There were no significantly downregulated genes in the vehicle RDEB skin compared to the WT. In the DCN-treated RDEB mouse skin, the overall gene expression profile was similar to that in the vehicle RDEB skin (FIG. 6B). Even though the expression of Tgfb1 was no longer significantly abnormal, the expression of Tgfbr3 and Ctgf was still significantly upregulated in the DCN treated RDEB skin. Some genes, such as Il4, Cxcl3, Tnfα were more significantly upregulated in the DCN-treated RDEB skin than in the vehicle control (FIG. 6B and Table 2).

Importantly, the expression profile of DCN-tCRK-treated RDEB skin was markedly different from those of vehicle and DCN-treated RDEB skin and resembled that of WT skin (FIG. 6A). Although it showed individual variation in the expression of some genes, none of the genes in the array were significantly dysregulated in DCN-tCRK treated RDEB skin when compared to the WT (FIG. 6 and Table 2).

TABLE 2
Fold changes of gene expression in vehicle, DCN and DCN-tCRK treated
col7a^{-/-} skin relative to the WT and P values. N/A indicates
average threshold cycle either not determined or greater than the
defined cut-off. The genes that are significantly upregulated as compared
to the WT are bolded and the genes that are significantly upregulated
only in DCN-treated col7a^{-/-} skin are underlined.

| | Vehicle RDEB | | DCN-tCRK RDEB | | DCN RDEB | |
|---|---|---|---|---|---|---|
| | vs. WT | | vs. WT | | vs. WT | |
| Gene | Fold | | Fold | | Fold | |
| Symbols | changes | P value | changes | P value | changes | P value |
| Acta2 | 1.55 | 0.695 | 0.84 | 0.672 | N/A | N/A |
| Actc1 | 1.19 | 0.633 | 1.36 | 0.815 | 0.65 | 0.426 |
| Angpt1 | 1.54 | 0.545 | 0.72 | 0.927 | 2.94 | 0.271 |
| Ccl12 | 8.28 | 0.375 | N/A | N/A | N/A | N/A |
| Ccl7 | 0.66 | 0.701 | 0.74 | 0.594 | 3.07 | 0.001 |
| Cd40lg | 0.85 | 0.783 | N/A | N/A | N/A | N/A |
| Cdh1 | 2.58 | 0.076 | 1.76 | 0.439 | 3.20 | 0.062 |
| Col14a1 | 0.89 | 0.690 | 0.84 | 0.737 | 1.02 | 0.751 |
| Col1a1 | 1.79 | 0.405 | 1.45 | 0.396 | 1.16 | 0.893 |
| Col1a2 | 0.82 | 0.897 | 0.53 | 0.830 | 1.2 | 0.722 |
| Col3a1 | 1.06 | 0.885 | 0.6 | 0.74 | 1.71 | 0.501 |
| Col4a1 | 0.94 | 0.487 | 0.25 | 0.356 | N/A | N/A |
| Col4a3 | 2.96 | 0.203 | 1.08 | 0.655 | 3.16 | 0.068 |
| Col5a1 | 4.47 | 0.195 | 1.21 | 0.582 | 1.12 | 0.226 |
| Col5a2 | 1.24 | 0.554 | 0.61 | 0.795 | 1.34 | 0.572 |
| Col5a3 | 1.86 | 0.370 | 1.8 | 0.410 | 1.83 | 0.228 |
| Csf2 | 0.91 | 0.849 | 0.7 | 0.762 | 45.86 | 0.004 |
| Csf3 | 1.36 | 0.439 | 2.19 | 0.332 | 7.04 | 0.049 |
| Ctgf | 3.26 | 0.037 | 2.44 | 0.281 | 4.55 | 0.050 |
| Ctnnb1 | 3.08 | 0.009 | 2.25 | 0.349 | 3.79 | 0.009 |
| Ctsg | 3.15 | 0.002 | 1.77 | 0.292 | 9.23 | 0.021 |
| Ctsk | 1.7 | 0.266 | 0.69 | 0.897 | 1.4 | 0.422 |
| Ctsl | 2.25 | 0.285 | 1.01 | 0.883 | 2.42 | 0.207 |
| Cxcl1 | 4.97 | 0.106 | 2.05 | 0.326 | 12.49 | 0.085 |
| Cxcl11 | N/A | N/A | N/A | N/A | N/A | N/A |
| Cxcl3 | 3.41 | 0.009 | 1.98 | 0.073 | 26.44 | 0.047 |
| Cxcl5 | 1.25 | 0.449 | 1.35 | 0.482 | 7.03 | 0.023 |
| Egf | 0.66 | 0.218 | 0.54 | 0.816 | 0.67 | 0.031 |
| Egfr | 4.68 | 0.015 | 1.75 | 0.367 | 2.20 | 0.039 |
| F13a1 | 1.96 | 0.493 | 0.82 | 0.747 | 1.78 | 0.484 |
| F3 | 3.04 | 0.114 | 1.74 | 0.423 | 2.99 | 0.172 |
| Fga | N/A | N/A | N/A | N/A | N/A | N/A |
| Fgf10 | 1.84 | 0.164 | 1.15 | 0.543 | 1.56 | 0.317 |
| Fgf2 | 2.2 | 0.125 | 1.17 | 0.633 | 3.49 | 0.164 |
| Fgf7 | 1.23 | 0.761 | 1.1 | 0.730 | 1.13 | 0.951 |
| Hbegf | 2.69 | 0.154 | 1.11 | 0.658 | 4.01 | 0.026 |
| Hgf | 3.36 | 0.377 | 0.25 | 0.259 | 8.12 | 0.374 |
| Ifng | N/A | N/A | N/A | N/A | N/A | N/A |
| Igf1 | 1.22 | 0.606 | 0.71 | 0.694 | 1.79 | 0.337 |
| Il10 | 2.00 | 0.408 | 1.1 | 0.979 | 3.26 | 0.235 |
| Il1b | 4.12 | 0.262 | 1.1 | 0.608 | 100.49 | 0.132 |
| Il2 | N/A | N/A | N/A | N/A | N/A | N/A |
| Il4 | 4.29 | 0.015 | 2.08 | 0.302 | 13.75 | 0.006 |
| Il6 | 1.62 | 0.358 | 1.34 | 0.571 | 8.75 | 0.082 |
| Il6st | 1.81 | 0.313 | 1.58 | 0.481 | 2.35 | 0.141 |
| Itga1 | 1.58 | 0.42 | 0.95 | 0.909 | 1.2 | 0.913 |
| Itga2 | 2.24 | 0.16 | 1.45 | 0.547 | 2.17 | 0.197 |
| Itga3 | 4.57 | 0.104 | 2.17 | 0.172 | 2.01 | 0.003 |
| Itga4 | 2.26 | 0.746 | 0.447 | 0.304 | N/A | N/A |
| Itga5 | 3.37 | 0.207 | 1.62 | 0.397 | 2.39 | 0.127 |
| Itga6 | 2.41 | 0.106 | 1.39 | 0.507 | 4.31 | 0.295 |
| Itgav | 2.25 | 0.148 | 1.32 | 0.557 | 2.14 | 0.159 |
| Itgb1 | 2.29 | 0.178 | 0.69 | 0.769 | 2.90 | 0.048 |
| Itgb3 | 1.57 | 0.001 | 0.57 | 0.084 | 1.24 | 0.391 |
| Itgb5 | 5.78 | 0.050 | 3.79 | 0.266 | 3.29 | 0.181 |
| Itgb6 | 1.26 | 0.228 | 0.62 | 0.680 | 2.68 | 0.329 |
| Mapk1 | 2.01 | 0.167 | 1.42 | 0.488 | 3.44 | 0.012 |
| Mapk3 | 2.18 | 0.040 | 1.33 | 0.504 | 1.75 | 0.267 |
| Mif | 0.4 | 0.709 | 1.64 | 0.439 | 1.1 | 0.986 |
| Mmp1a | 1.27 | 0.451 | 0.95 | 0.713 | 0.26 | 0.013 |
| Mmp2 | N/A | N/A | N/A | N/A | N/A | N/A |
| Mmp7 | N/A | N/A | N/A | N/A | N/A | N/A |
| Mmp9 | 1.18 | 0.879 | 0.71 | 0.810 | 2.33 | 0.355 |
| Pdgfa | 1.27 | 0.785 | 1.17 | 0.587 | 4.35 | 0.065 |
| Plat | 1.31 | 0.603 | 1.41 | 0.416 | 0.93 | 0.814 |
| Plaur | 3.59 | 0.002 | 2.72 | 0.342 | 20.26 | 0.041 |
| Plau | 2.41 | 0.127 | 1.39 | 0.464 | 3.94 | 0.122 |
| Plg | N/A | N/A | N/A | N/A | N/A | N/A |
| Pten | 3.79 | 0.203 | 0.605 | 0.159 | 4.59 | 4.59 |
| Ptgs2 | 1.84 | 0.79 | 0.435 | 0.318 | 8.44 | 0.167 |
| Rac1 | 1.42 | 0.860 | 0.94 | 0.693 | 0.65 | 0.752 |
| Rhoa | 3.54 | 0.101 | 2.97 | 0.376 | 7.69 | 0.043 |
| Serpine1 | 5.63 | 0.111 | 2.42 | 0.309 | 5.00 | 0.062 |
| Stat3 | 3.8 | 0.065 | 2.19 | 0.377 | 4.65 | 0.172 |
| Tagln | 1.32 | 0.156 | 0.56 | 0.801 | 0.78 | 0.908 |
| Tgfa | N/A | N/A | N/A | N/A | N/A | N/A |
| Tgfb1 | 2.38 | 0.040 | 1.6 | 0.332 | 2.33 | 0.361 |
| Tgfbr3 | 8.11 | 0.006 | 3.93 | 0.222 | 6.46 | 0.041 |
| Timp1 | 0.29 | 0.194 | 0.22 | 0.168 | 2.25 | 0.331 |
| Tnf | 3.92 | 0.024 | 1.1 | 0.683 | 12.16 | 0.049 |
| Vegfa | 6.54 | 0.129 | 3.02 | 0.369 | 3.4 | 0.274 |
| Vtn | 1.22 | 0.721 | 0.75 | 0.985 | 1.23 | 0.780 |
| Wisp1 | 1.26 | 0.693 | 0.70 | 0.890 | 1.23 | 0.781 |
| Wnt5a | 1.89 | 0.212 | 1.32 | 0.493 | 1.08 | 0.895 |

Supporting the development of TGFβ1-mediated fibrosis in untreated RDEB skin and its suppression by DCN-tCRK treatment, strong expression of CTGF/CCN2 was observed in vehicle-injected RDEB skin and the expression level was markedly diminished after treatment with DCN-tCRK (FIG. 7A). Moreover, the overall collagen deposition increased with time in the vehicle-injected RDEB skin, as demonstrated by picrosirius red staining, but was significantly decreased in the DCN-tCRK treated mouse skin (FIGS. 7B and 7C). Immunostaining demonstrated a substantial increase in the expression of type I collagen (COL1) in the vehicle-treated skin and an attenuated expression in the DCN-tCRK treated skin at the two-week time point (FIGS. 7D and 7E). Similar results were obtained with immunostaining of myofibroblasts, i.e. α-smooth muscle actin (αSMA, FIG. 7D ja 7E). Moreover, most of the αSMA+ cells in the WT as well as DCN-tCRK treated RDEB skin co-localized with blood vessels (CD31 staining), which indicates their identity as blood vessel smooth muscle cells and pericytes, whereas the αSMA+ cells in the vehicle-treated RDEB skin were outside of the blood vessels, i.e. indicative of being myofibroblasts (FIG. 7D).

To directly demonstrate the anti-fibrotic function of DCN-tCRK, the abilities of DCN and DCN-tCRK to suppress the collagen gel contraction in vitro were compared, using both normal and RDEB-derived fibroblasts. At a low concentration (75 μM) that DCN had no significant effect on collagen contraction, DCN-tCRK suppressed the collagen gel contraction in both normal (p<0.05) and RDEB-derived (p<0.01) fibroblasts (FIG. 8).

### Discussion

It is demonstrated herein that a C-terminal exposure of CendR sequence in a wound homing peptide renders a novel tissue penetrating function of the peptide in normal and wounded skin. Conjugation of tCRK peptide to DCN facilitates skin-selective targeting of the therapeutic fusion protein that exerts anti-fibrotic effects and improves survival in a murine model of RDEB.

The experiments demonstrated that systemic administration of DCN-tCRK recombinant protein was more effective than unmodified DCN in improving the survival of col7a1^{-/-} mice. The exact molecular mechanism is not known, but it is assumed, without being limited to any theory, that multiple different mechanisms could contribute to the improved survival. DCN is an anti-inflammatory and -fibrotic molecule. Consistent with the Inventors' previous finding on the activation of TGFβ signaling as early as a week after birth, the expression of more than half of the genes related to fibrosis formation were up-regulated in the untreated RDEB mouse skin at the one-week time point. Without being limited to any theory, the improved survival of RDEB mice by DCN-tCRK administration is likely related to the anti-fibrotic and anti-inflammatory effects of the therapeutic protein.

Not only the genes directly involved in TGFβ signaling were normalized in the DCN-tCRK (but not in DCN) treated RDEB skin, the genes related to other signaling pathways, such as β-catenin and EGFR, were also normalized by DCN-tCRK administration. Both Wnt/β-catenin and EGFR signaling have been demonstrated to contribute to fibrogenesis in multiple fibrotic diseases through their independent, profibrotic mechanisms or via cross-talking with the TGFβ signaling. For example, EGFR activation is required for profibrotic functions of TGFβ and CCN2-mediated fibroblast proliferation and myofibroblast transdifferentiation. DCN can bind and downregulate EGFR and HGF receptor Met (to suppress expression of β-catenin). The normalized expression of these genes in the col7a1^{-/-} mouse skin after administration of DCN-tCRK suggests multiple therapeutic functions of DCN-tCRK in RDEB. The up-regulation of pro-inflammatory genes in DCN treated RDEB skin, in turn, may indicate therapeutic effect that was not sustained by the administration of native DCN.

In summary, it is demonstrated herein that exposure of a cryptic CendR sequence renders novel features in a wound targeting peptide to home to normal skin in addition to the wounded skin and also provides dermal tissue penetration. It is also demonstrated that this peptide (tCRK) can serve as a vehicle for delivering decorin and other therapeutic molecules in the treatment of systemic dermal diseases, especially epidermolysis bullosa.

1. A homing peptide-guided decorin conjugate for use in the treatment of epidermolysis bullosa, wherein the conjugate comprises a decorin segment and a homing peptide, wherein the C-terminal end of the homing peptide consists of the amino acid sequence RKDK (SEQ ID NO: 1) or CRKDK (SEQ ID NO: 2).

2. The conjugate for use according to claim 1, wherein the conjugate selectively homes to skin and skin wounds.

3. The conjugate for use according to claim 1, wherein the decorin segment is attached to the N-terminal end of the homing peptide.

4. The conjugate for use according to claim 1, wherein the decorin segment comprises an amino acid sequence having at least 80% sequence identity with the amino acid sequence set forth in any one of SEQ ID NOs: 6-20, or is a conservative sequence variant or a peptidomimetic of said decorin segment, provided that biological properties of decorin are retained.

5. The conjugate for use according to claim 1, wherein the conjugate is a fusion protein or a peptidomimetic thereof.

6. The conjugate for use according to claim 5, wherein the fusion protein comprises or consists of SEQ ID NO: 21 or 22.

7. The conjugate for use according to claim 1, wherein the conjugate further comprises one or more additional moieties attached to the conjugate.

8. The conjugate for use according to claim 7, wherein said one or more additional moieties comprise a therapeutic agent.

9. The conjugate for use according to claim 8, wherein the therapeutic agent is an anti-inflammatory agent, an anti-angiogenic agent, a regenerative agent, a pro-angiogenic agent, a cytotoxic agent, a pro-apoptotic agent, an antimicrobial agent, an anti-fibrotic agent, an anti-wrinkle agent, an anti-itch agent, an anti-transmitter agent, a pro-transmitter agent, a cytokine, or a cytokine inhibitor.

10. The conjugate for use according to claim 9, wherein the therapeutic agent is a peptide, a polypeptide, a protein, or a small molecule.

11. The conjugate for use according to claim 7, wherein said one or more additional moieties comprise a detectable agent.

12. The conjugate for use according to claim 1, wherein the conjugate is provided in a pharmaceutical composition comprising the conjugate and a pharmaceutically acceptable carrier.

13. The conjugate for use according to claim 1, wherein epidermolysis bullosa is acquired epidermolysis bullosa, junctional epidermolysis bullosa, epidermolysis bullosa simplex, dystrophic epidermolysis bullosa, dominant dystrophic epidermolysis bullosa, recessive dystrophic epidermolysis bullosa, recessive dystrophic epidermolysis bullosa inversa, Kindler syndrome or any subtype thereof.


SEQUENCE LISTING (26 sequences)

SEQ ID NO: 1 - protein, 4 aa, Homo sapiens
RKDK

SEQ ID NO: 2 - protein, 5 aa, Homo sapiens
CRKDK

SEQ ID NO: 3 - protein, 6 aa, Homo sapiens
CRKDKC

SEQ ID NO: 4 - protein, 4 aa, Homo sapiens
  MISC_FEATURE (1)..(1): Xaa is Arg or Lys
  MISC_FEATURE (2)..(2): Xaa is any amino acid
  MISC_FEATURE (3)..(3): Xaa is any amino acid
  MISC_FEATURE (4)..(4): Xaa is Arg or Lys
XXXX

SEQ ID NO: 5 - protein, 9 aa, Homo sapiens
CARSKNKDC

SEQ ID NO: 6 - protein, 358 aa, Homo sapiens
MKATIILLLLAQVSWAGPFQQRGLFDFMLEDEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 7 - protein, 342 aa, Homo sapiens
GPFQQRGLFDFMLEDEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 8 - protein, 312 aa, Homo sapiens
EPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 9 - protein, 250 aa, Homo sapiens
MKATIILLLLAQVSWAGPFQQRGLFDFMLEDEASGIGPEVPDDRDFEPSLGPVCPFRCQCHLRVVQCSDLELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 10 - protein, 234 aa, Homo sapiens
GPFQQRGLFDFMLEDEASGIGPEVPDDRDFEPSLGPVCPFRCQCHLRVVQCSDLELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 11 - protein, 204 aa, Homo sapiens
EPSLGPVCPFRCQCHLRVVQCSDLELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 12 - protein, 212 aa, Homo sapiens
MKATIILLLLAQVSWAGPFQQRGLFDFMLEDEASGIGPEVPDDRDFEPSLGPVCPFRCQCHLRVVQCSDLGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 13 - protein, 196 aa, Homo sapiens
GPFQQRGLFDFMLEDEASGIGPEVPDDRDFEPSLGPVCPFRCQCHLRVVQCSDLGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 14 - protein, 166 aa, Homo sapiens
EPSLGPVCPFRCQCHLRVVQCSDLGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 15 - protein, 172 aa, Homo sapiens
MKATIILLLLAQVSWAGPFQQRGLFDFMLEDEASGIGPEVPDDRDFEPSLGPVCPFRCQCHLRVVQCSDLGLDKVPKDLPPDTTLLDLQNNKITEIKDGDFKNLKNLHVVYLHNNNISVVGSSDFCPPGHNTKKASYSGVSLFSNPVQYWEIQPSTFRCVYVRSAIQLGNYK

SEQ ID NO: 16 - protein, 156 aa, Homo sapiens
GPFQQRGLFDFMLEDEASGIGPEVPDDRDFEPSLGPVCPFRCQCHLRVVQCSDLGLDKVPKDLPPDTTLLDLQNNKITEIKDGDFKNLKNLHVVYLHNNNISVVGSSDFCPPGHNTKKASYSGVSLFSNPVQYWEIQPSTFRCVYVRSAIQLGNYK

SEQ ID NO: 17 - protein, 126 aa, Homo sapiens
EPSLGPVCPFRCQCHLRVVQCSDLGLDKVPKDLPPDTTLLDLQNNKITEIKDGDFKNLKNLHVVYLHNNNISVVGSSDFCPPGHNTKKASYSGVSLFSNPVQYWEIQPSTFRCVYVRSAIQLGNYK

SEQ ID NO: 18 - protein, 75 aa, Homo sapiens
MKATIILLLLAQVSWAGPFQQRGLFDFMLEDEASGIGPEVPDDRDFEPSLGPVCPFRCQCHLRVVQCSDLGCLPS

SEQ ID NO: 19 - protein, 59 aa, Homo sapiens
GPFQQRGLFDFMLEDEASGIGPEVPDDRDFEPSLGPVCPFRCQCHLRVVQCSDLGCLPS

SEQ ID NO: 20 - protein, 29 aa, Homo sapiens
EPSLGPVCPFRCQCHLRVVQCSDLGCLPS

SEQ ID NO: 21 - protein, 345 aa, Artificial Sequence
  Decorin-tCRK fusion
GHHHHHHDEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYKGSEFCRKDK

SEQ ID NO: 22 - protein, 368 aa, Artificial Sequence
  Decorin-tCRK fusion
MKATIILLLLAQVSWAGPFQQRGLFDFMLEDEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYKGSEFCRKDK

SEQ ID NO: 23 - protein, 4 aa, Artificial Sequence
  Peptide linker
GSEF

SEQ ID NO: 24 - protein, 5 aa, Artificial Sequence
  Peptide linker
GSEFC

SEQ ID NO: 25 - protein, 7 aa, Artificial Sequence
  Synthetic peptide
RPARPAR

SEQ ID NO: 26 - protein, 8 aa, Artificial Sequence
  Synthetic peptide
RPARPARA